(19) United States
(12) Patent Application Publication
(10) Pub. No.: US 2011/0269786 A1
(43) Pub. Date: Nov. 3, 2011

(54) TREATMENT OF MAJOR ADVERSE CARDIAC EVENTS AND ACUTE CORONARY SYNDROME IN DIABETIC PATIENTS USING SECRETORY PHOSPHOLIPASE A2 (SPLA2) INHIBITOR OR SPLA2 INHIBITOR COMBINATION THERAPIES

(76) Inventors: Colin Hislop, Menlo Park, CA (US); Joaquim Trias, Millbrae, CA (US); Debra Odink, Oakland, CA (US); Paul Truex, Pleasanton, CA (US)

(21) Appl. No.: 13/098,261

(22) Filed: Apr. 29, 2011

Related U.S. Application Data
(60) Provisional application No. 61/329,976, filed on Apr. 30, 2010.

(51) Int. Cl. A61K 31/435; A61K 31/47; A61P 9/10; A61K 31/404

(52) U.S. Cl. 514/277; 514/419; 514/311

(57) ABSTRACT

Administration of sPLA₂ inhibitors in combination with statins has been found to reduce the occurrence of major adverse cardiac events (MACEs), specifically unstable angina (UA) requiring urgent hospitalization, in diabetic subjects who have recently experienced an index ACS event to a significantly greater degree than statins alone. These results were unexpected given previous results showing that statins alone are insufficient to satisfactorily reduce MACEs and inflammation in this high-risk population, combined with the high levels of baseline inflammation associated with diabetes. Therefore, provided herein are methods of treating MACEs, including UA requiring urgent hospitalization, in a diabetic subject who has previously experienced an ACS event by administering one or more sPLA₂ inhibitors alone or in combination with one or more statins.

### RELATED APPLICATIONS

The present utility application claims priority to U.S. Provisional Patent Application No. 61/329,976, filed Apr. 30, 2010, the disclosure of which is incorporated by reference herein in its entirety.

### BACKGROUND

Coronary occlusion arising from thrombus formation results in acute coronary syndrome (ACS), a set of ischemic conditions that include unstable angina (UA), non-ST-segment elevation myocardial infarction (NSTEMI), and ST-segment elevation myocardial infarction (STEMI). UA and NSTEMI are generally associated with nonocclusive or partially occlusive thrombus formation, whereas STEMI results from a more stable occlusive thrombus. UA and NSTEMI are closely related and have very similar clinical presentations. ACS events affect approximately 1.4 million people in the United States annually as 700,000 new events, 500,000 recurrent events, and 175,000 silent events.

ACS events are following by an acute inflammatory response and decreased plaque stability. This inflammatory response places post-ACS subjects at a very high risk of major adverse cardiac events (MACEs) during the months following the ACS event. This risk is particularly acute in post-ACS subjects with diabetes, since diabetes is associated with high baseline levels of inflammation. Therefore, there is a need for new methods of preventing MACE occurrence in acute post-ACS diabetic patients.

### SUMMARY

In certain embodiments, methods are provided for treating a MACE in a diabetic subject who has previously experienced an ACS event by administering a therapeutically effective amount of one or more sPLA₂ inhibitors and a therapeutically effective amount of one or more statins. In certain of these embodiments, the previous ACS event occurred or was diagnosed within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain embodiments, the one or more sPLA₂ inhibitors include A-001 or a pharmaceutically acceptable salt, solvate, or prodrug thereof. In certain embodiments the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain embodiments, the one or more statins include atorvastatin, rosuvastatin, simvastatin, lovastatin, pravastatin, cerivastatin, fluvastatin, mevastatin, pitavastatin, and/or a statin combination drug. In certain of these embodiments, administration of one or more sPLA₂ inhibitors in combination with one or more statins treats the MACE more effectively than administration of the one or more statins alone. In certain embodiments, the MACE is UA (including UA requiring urgent hospitalization). In other embodiments, the MACE is cardiovascular death, fatal or non-fatal MI, fatal or non-fatal stroke, and/or risk of or danger associated with revascularization. In certain embodiments, treatment of a MACE prevents the MACE, reduces the likelihood of occurrence of the MACE, delays the occurrence of the MACE, and/or decreases the severity of the MACE. In certain embodiments, the one or more sPLA₂ inhibitors are administered at regular intervals for a time period of 24 weeks or less, 20 weeks or less, 16 weeks or less, 12 weeks or less, 8 weeks or less, 4 weeks or less, or 2 weeks or less.

In certain embodiments, methods are provided for inhibiting inflammation in a diabetic subject who has previously experienced an ACS event by administering a therapeutically effective amount of one or more sPLA₂ inhibitors and a therapeutically effective amount of one or more statins. In certain of these embodiments, the previous ACS event occurred or was diagnosed within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain embodiments, the one or more sPLA₂ inhibitors include A-001 or a pharmaceutically acceptable salt, solvate, or prodrug thereof. In certain embodiments the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain embodiments, the one or more statins include atorvastatin, rosuvastatin, simvastatin, lovastatin, pravastatin, cerivastatin, fluvastatin, mevastatin, pitavastatin, and/or a statin combination drug. In certain embodiments, the one or more sPLA₂ inhibitors are administered at regular intervals for a time period of 24 weeks or less, 20 weeks or less, 16 weeks or less, 12 weeks or less, 8 weeks or less, 4 weeks or less, or 2 weeks or less.

In certain embodiments, the use of one or more sPLA₂ inhibitors as an adjunct to statins to treat a MACE in a diabetic subject who has previously experienced an ACS event is provided. In certain of these embodiments, the previous ACS event occurred or was diagnosed within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain embodiments, the one or more sPLA₂ inhibitors include A-001 or a pharmaceutically acceptable salt, solvate, or prodrug thereof. In certain embodiments, the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain embodiments, the statin is atorvastatin, rosuvastatin, simvastatin, lovastatin, pravastatin, cerivastatin, fluvastatin, mevastatin, pitavastatin, and/or a statin combination drug. In certain embodiments, treatment of a MACE prevents the MACE, reduces the likelihood of occurrence of the MACE, delays the occurrence of the MACE, and/or decreases the severity of the MACE. In certain of these embodiments, administration of one or more sPLA₂ inhibitors in combination with statin is more effective at preventing MACEs than administration of statin alone. In certain embodiments, the MACE being prevented is UA (including UA requiring urgent hospitalization). In other embodiments, the MACE is cardiovascular death, fatal or non-fatal MI, fatal or non-fatal stroke, and/or risk of or danger associated with revascularization. In certain embodiments, the one or more sPLA₂ inhibitors are administered at regular intervals for a time period of 24 weeks or less, 20 weeks or less, 16 weeks or less, 12 weeks or less, 8 weeks or less, 4 weeks or less, or 2 weeks or less.

In certain embodiments, methods are provided for increasing the effectiveness of one or more statins for the treatment of MACEs in a diabetic subject who has previously experienced an ACS event by administering a therapeutically effective amount of one or more sPLA₂ inhibitors. In certain of these embodiments, the previous ACS event occurred or was diagnosed within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain embodiments, the one or more sPLA₂ inhibitors include A-001 or a pharmaceutically acceptable salt, solvate, or prodrug thereof. In certain embodiments, the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain embodiments, the MACE being treated is UA (including UA requiring urgent hospitalization). In other embodiments, the MACE is cardiovascular death, fatal or non-fatal MI, fatal or non-fatal stroke, and/or risk of or danger associated with revascularization. In certain embodiments, the one or more statins include atorvastatin, rosuvastatin, simvastatin, lovastatin, pravastatin, cerivastatin, fluvastatin, mevastatin, pitavastatin, and/or a statin combination drug. In certain embodiments, the one or more sPLA₂ inhibitors are administered at regular intervals for a time period of 24 weeks or less, 20 weeks or less, 16 weeks or less, 12 weeks or less, 8 weeks or less, 4 weeks or less, or 2 weeks or less.

In certain embodiments, compositions and kits are provided for treating MACEs and/or inhibiting inflammation in a diabetic subject who has previously experienced an ACS event. These kits contain one or more sPLA₂ inhibitors, and optionally contain one or more statins. In certain embodiments, the kits further contain instructions for usage. In those embodiments, where the kits contain both one or more sPLA₂ inhibitors and one or more statins, the one or more sPLA₂ inhibitors and one or more statins may be contained a single packaging unit within the kit (e.g., in a single bottle or blister pack) or in separate packaging units. In certain embodiments, the previous ACS event occurred or was diagnosed within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain embodiments, the one or more sPLA₂ inhibitors include A-001 or a pharmaceutically acceptable salt, solvate, or prodrug thereof. In certain embodiments, the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In those embodiments where the kit contains one or more statins, the one or more statins may be selected from atorvastatin, rosuvastatin, simvastatin, lovastatin, pravastatin, cerivastatin, fluvastatin, mevastatin, pitavastatin, and/or a statin combination drug. In certain embodiments, the MACE being treated is UA (including UA requiring urgent hospitalization). In other embodiments, the MACE is cardiovascular death, fatal or non-fatal MI, fatal or non-fatal stroke, and/or risk of or danger associated with revascularization.

In certain embodiments, the use of one or more sPLA₂ inhibitors and one or more statins for producing a medicament for use in the treatment of MACEs in a diabetic subject who has previously experienced an ACS event is provided. In certain of these embodiments the previous ACS event occurred or was diagnosed within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain embodiments, the one or more sPLA₂ inhibitors include A-001 or a pharmaceutically acceptable salt, solvate, or prodrug thereof. In certain embodiments, the prodrug of A-001 is a C₁-C₆ alkyl ester prodrug, an acyloxyalkyl ester prodrug, or an alkyloxycarbonyloxyalkyl ester prodrug, and in certain of these embodiments the prodrug is A-002. In certain of these embodiments, the one or more statins are atorvastatin, rosuvastatin, simvastatin, lovastatin, pravastatin, cerivastatin, fluvastatin, mevastatin, pitavastatin, and/or a statin combination drug. In certain embodiments, the MACE being treated is UA (including UA requiring urgent hospitalization). In other embodiments, the MACE is cardiovascular death, fatal or non-fatal MI, fatal or non-fatal stroke, and/or risk of or danger associated with revascularization.

### BRIEF DESCRIPTION OF DRAWINGS

FIG. 1: Effect of A-002 administration on serum hs-CRP levels in diabetes subpopulation at weeks 2, 4, 8, 16, and 24. ♦=500 mg A-002 plus 80 mg atorvastatin (“A-002”); ▪=80 mg atorvastatin only (“Placebo”).

FIG. 2: Effect of A-002 administration on serum IL-6 levels in diabetes subpopulation at weeks 2, 4, and 8. ♦=500 mg A-002 plus 80 mg atorvastatin (“A-002”); ▪=80 mg atorvastatin only (“Placebo”).

FIG. 3: Effect of A-002 administration on composite MACE occurrence in diabetes subpopulation. Number below days on X-axis indicates the number of subjects at each timepoint.

### DETAILED DESCRIPTION

The following description of the invention is merely intended to illustrate various embodiments of the invention. As such, the specific modifications discussed are not to be construed as limitations on the scope of the invention. It will be apparent to one skilled in the art that various equivalents, changes, and modifications may be made without departing from the scope of the invention, and it is understood that such equivalent embodiments are to be included herein.

### Abbreviations

ACS, acute coronary syndrome; BMI, body mass index; CAD, coronary artery disease; CHD, coronary heart disease; CK, cardiac troponin; CVD, cardiovascular disease; ECG, electrocardiogram; hs-CRP, high-sensitivity C-reactive protein; LDL or LDL-C, low density lipoprotein; MACE, major adverse cardiac event; MI, myocardial infarction; NSTEMI, non-ST-segment elevation myocardial infarction; sPLA₂, secretory phospholipase A₂; STEMI, ST-segment elevation myocardial infarction; t_{1/2}, terminal half-life; TG, triglyceride; UA, unstable angina; ULN, upper limit of normal.

The term “subject” as used herein refers to any mammal, preferably a human. A “diabetic subject” or “subject with diabetes” is any subject that has been diagnosed with either type I or type II diabetes.

A “subject in need thereof” refers to a diabetic subject who has previously experienced an ACS event.

An “ACS event” or “index ACS event” as used herein refers to UA, NSTEMI, or STEMI.

“Angina” as used herein refers generally to chest pain caused by poor blood flow and corresponding decreased oxygen delivery to the heart. Stable or chronic angina occurs only during activity or stress. UA, on the other hand, can occur suddenly without cause. Subjects with angina are at increased risk for MI.

A “major adverse cardiac event” or “MACE” as used herein includes UA, cardiovascular death, fatal or non-fatal MI, fatal or non-fatal stroke, need for a revascularization procedure, heart failure, resuscitated cardiac arrest, and/or new objective evidence of ischemia, as well as any and all subcategories of events falling within each of these event types (e.g., STEMI and NSTEMI, documented UA requiring urgent hospitalization). In certain embodiments, MACE refers specifically to UA requiring urgent hospitalization, cardiovascular death, non-fatal MI, non-fatal stroke, and/or need for revascularization procedure.

The term “statin” as used herein refers to any compound that inhibits HMG-CoA reductase, an enzyme that catalyzes the conversion of HMG-CoA to mevalonate.

The terms “treat,” “treating,” or “treatment” as used herein generally refer to preventing a condition or event, slowing the onset or rate of development of a condition or delaying the occurrence of an event, reducing the risk of developing a condition or experiencing an event, preventing or delaying the development of symptoms associated with a condition or event, reducing or ending symptoms associated with a condition or event, generating a complete or partial regression of a condition, lessening the severity of a condition or event, or some combination thereof.

With regard to MACEs, the terms “treat,” “treating,” or “treatment” refer to preventing MACEs or MACE recurrence, reducing the likelihood of MACEs or MACE recurrence, delaying the occurrence of MACEs, reducing the severity of MACEs or one or more symptoms associated with MACEs, and/or preventing, delaying or reducing the development of one or more symptoms related to MACEs. For each of these, the effect on MACEs may refer to an effect on MACEs generally (e.g., a reduction in the likelihood of occurrence of all types of MACE), an effect on one or more specific types of MACE (e.g., a reduction in the likelihood of UA requiring urgent hospitalization), or a combination thereof. In those cases where treatment refers to an effect on one or more specific MACEs, treatment may result in a decrease in the likelihood or severity of one or more types of MACEs without exhibiting an effect on MACEs generally.

A “therapeutically effective amount” of a composition as used herein is an amount of a composition that produces a desired therapeutic effect in a subject, such as treating a target condition. The precise therapeutically effective amount is an amount of the composition that will yield the most effective results in terms of therapeutic efficacy in a given subject. This amount will vary depending upon a variety of factors, including but not limited to the characteristics of the therapeutic composition (including, e.g., activity, pharmacokinetics, pharmacodynamics, and bioavailability), the physiological condition of the subject (including, e.g., age, body weight, sex, disease type and stage, medical history, general physical condition, responsiveness to a given dosage, and other present medications), the nature of the pharmaceutically acceptable carrier or carriers in the composition, and the route of administration. One skilled in the clinical and pharmacological arts will be able to determine a therapeutically effective amount through routine experimentation, namely by monitoring a subject's response to administration of a composition and adjusting the dosage accordingly. For additional guidance, see, e.g., Remington: The Science and Practice of Pharmacy, 21^{st} Edition, Univ. of Sciences in Philadelphia (USIP), Lippincott Williams & Wilkins, Philadelphia, Pa., 2005, and Goodman & Gilman's The Pharmacological Basis of Therapeutics, 11th Edition, McGraw-Hill, New York, N.Y., 2006, the entire disclosures of which are incorporated by reference herein.

A “pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. Such a carrier may comprise, for example, a liquid, gel, solid, or semi-solid filler, solvent, surfactant, diluent, excipient, adjuvant, binder, buffer, dissolution aid, solvent, encapsulating material, sequestering agent, dispersing agent, preservative, lubricant, disintegrant, thickener, emulsifier, antimicrobial agent, antioxidant, stabilizing agent, coloring agent, flavoring agent, or some combination thereof. Each component of the carrier must be “pharmaceutically acceptable” in that it must be compatible with the other ingredients of the composition and must be suitable for contact with any tissue, organ, or portion of the body that it may encounter, meaning that it must not carry a risk of toxicity, irritation, allergic response, immunogenicity, or any other complication that excessively outweighs its therapeutic benefits. Examples of pharmaceutically acceptable carriers for use in the presently disclosed pharmaceutical compositions include, but are not limited to, diluents such as microcrystalline cellulose or lactose (e.g., anhydrous lactose, lactose fast flo), binders such as gelatin, polyethylene glycol, wax, microcrystalline cellulose, synthetic gums such as polyvinylpyrrolidone, or cellulosic polymers such as hydroxypropyl cellulose (e.g., hydroxypropyl methylcellulose (HPMC)), lubricants such as magnesium stearate, calcium stearate, stearic acid, or microcrystalline cellulose, disintegrants such as starches, cross-linked polymers, or celluloses (e.g., croscarmellose sodium (CCNa), fillers such as silicon dioxide, titanium dioxide, microcrystalline cellulose, or powdered cellulose, surfactants or emulsifiers such as polysorbates (e.g., Polysorbate 20, 40, 60, or 80; Span 20, 40, 60, 65, or 80), antioxidant agents such as butylated hydroxyanisole (BHA), butylated hydroxytoluene (BHT), propyl gallate, or ascorbic acid (either free acid or salt forms thereof), buffers such as phosphate or citrate buffers, sequestering agents such as ethylenediaminetetraacetic acid (EDTA), ethylene glycol tetraacetic acid (EGTA), or edetate disodium, dispersing agents such as sodium carboxymethylcelluose, hydroxypropyl methylcellulose, povidone, or polyvinylpyrrolidone, dissolution aids such as calcium carbonate, and excipients such as water, saline, dextrose, glycerol, or ethanol, citric acid, calcium metabisulfite, lactic acid, malic acid, succinic acid, or tartaric acid.

Reducing cholesterol levels, particularly LDL-C levels, is currently the most common approach for treating CVD and conditions associated therewith. The goal of lowering cholesterol levels is to delay or reverse the onset and progression of atherosclerosis. In stable subjects, vessel narrowing due to atherosclerotic plaque formation is the primary cause of ischemic events such as MI or stroke. Lowering cholesterol levels in these stable subjects prevents additional plaque build-up, thereby reducing the risk and slowing the development CAD and CHD.

Among the most well-known and commonly used compounds for reducing cholesterol levels are statins. Statins inhibit HMG-CoA reductase from catalyzing the conversion of HMG-CoA to mevalonate, a rate-limiting step in the cholesterol biosynthetic pathway. As such, statins inhibit cholesterol biosynthesis and prevent the build-up of arterial plaque. Statin administration has been shown to lower both LDL-C and TG levels, and statins have also been shown to reduce inflammation and decrease blood levels of the inflammatory marker hs-CRP. Statins are routinely administered to stable subjects with chronic hyperlipidemia or established CVD, and have been shown to reduce cardiovascular events to some extent in stable populations with elevated cholesterol levels. In addition, recent studies have shown that statin administration to healthy subjects exhibiting elevated hs-CRP levels without hyperlipidemia lowers LDL-C and hs-CRP levels and decreases the risk of MACEs (Ridker 2008). However, statins are not always effective at preventing cardiovascular events. For example, 60-70% of cardiovascular events continue to occur despite statin therapy (Ridker 2005).

CHD and CAD are no longer viewed simply as lipid diseases, but also as complex inflammatory conditions. Inflammation contributes to atherosclerotic plaque build-up, and also plays a key role in the loss of collagen in the fibrous cap overlying atherosclerotic plaques. This loss of collagen decreases plaque stability, which in turn increases the likelihood of coronary thrombosis, a primary proximate cause of many MACEs. Since reduction of cholesterol levels is insufficient to prevent plaque instability, standard cholesterol-lowering therapies are not necessarily sufficient to treat CHD or CAD.

The danger associated with plaque instability is particularly high in unstable subjects who have recently experienced an ACS event (e.g., subjects who have experienced one or more ACS events or been diagnosed with one or more ACS events within the past 96 hours). ACS events are following by an acute inflammatory response, which is reflected by a short-term spike in levels of inflammatory markers such as hs-CRP, sPLA₂, and IL-6, as well as a marked decrease in plaque stability. Substantial elevations in sPLA₂ activity are generally observed within 24 hours of an ACS event, and this increased activity can continue for up to 12 weeks after the event. Inflammatory marker levels eventually drop back to pre-ACS event baseline levels, but subjects are at a very high risk of MACEs during the months following the event. This is especially true in diabetic subjects, who generally have high baseline levels of inflammation even before suffering an ACS event.

During the period following an ACS event, the ideal therapeutic approach is one that rapidly decreases inflammation, lowers cholesterol levels, prevents plaque build-up, and restores stability. Statins are routinely administered to the unstable post-ACS event population, but statin therapy alone is insufficient to prevent MACEs in these subjects. 15% of subjects who have recently experienced an ACS event and are treated with statin die or experience MI, stroke, or UA within four months after the initial event, and 22% experience these MACEs or require percutaneous coronary intervention (PCI) within two years (Schwartz 2005). Similar therapy data from the PROVE-IT TIMI-22 study demonstrated a 25% recurrent event rate at 2.5 years (Cannon 2004; Ridker 2005).

Phospholipases A₂ are a class of enzymes that play a role in inflammation by hydrolyzing the sn-2 fatty acyl chain of glycerophospholipids to produce lysophospholipids, resulting in downstream production of arachidonic acid, prostaglandins, and leukotrienes. The classes of phospholipase A₂ in humans include secretory phospholipase A₂ (sPLA₂) types IB, IIA, ITC, IID, IIE, IIF, III, V, X, and XII, lipoprotein-associated phospholipase A₂ (Lp-PLA₂, also known as PLA₂ type VII), cytosolic phospholipase (cPLA₂), and calcium-independent phospholipase A₂ (iPLA₂). Elevated levels of sPLA₂ types IIA, IID, IIE, IIF, III, V, and X have been observed in all stages of atherosclerosis development and have been implicated in atherogenesis based on their ability to degrade phospholipid (Kimura-Matsumoto 2007). sPLA₂ type IIA has been found to be expressed at vascular smooth muscle cells and foam cells in human arteriosclerosis lesions, and this expression has been correlated to the development of arteriosclerosis (Menschikowski 1995; Elinder 1997; Hurt-Camejo 1997). Transgenic mice that express high levels of human type IIA sPLA₂ have increased LDL-C levels, decreased HDL levels, decreased LDL-C and HDL particle size, and exhibit arteriosclerotic lesions (Ivandic 1999; Tietge 2000), and develop arteriosclerosis at a higher rate compared to normal mice when given a high fat diet (Ivandic 1999). Treatment with sPLA₂ modifies LDL-C lipoproteins such that they have higher affinity for extracellular matrix proteins (Camejo 1998; Sartipy 1999; Hakala 2001), resulting in an increased retention of LDL-C particles in the arterial wall. sPLA₂ treatment also reduces approximately 50% of the phospholipid moiety of normal LDL-C, resulting in smaller and denser particles that are more likely to form non-soluble complexes with proteoglycans and glycosaminoglycans (Sartipy 1999). In addition, there is some evidence that sPLA₂ remodels HDL, resulting in HDL catabolism (Pruzanski 1998). sPLA₂ type V is present in atherosclerotic lesions associated with smooth muscle cells and in surrounding foam cells in lipid core areas of the plaque in mice and humans (Rosengren 2006). sPLA₂ type V has been shown to increase arteriosclerosis in mice, while a deficiency of sPLA₂ type V has been shown to reduce arteriosclerosis (Rosengren 2006; Bostrom 2007). Lp-PLA2 is highly expressed in the necrotic core of coronary lesions (Serruys 2008).

sPLA₂ expression has also been correlated with an increased risk of development of CAD. Higher circulating levels of sPLA₂, and of sPLA₂ type IIA specifically, have been observed in patients with documented CAD than in control patients (Kugiyama 1999; Liu 2003; Boekholdt 2005; Chait 2005; Hartford 2006). In addition, higher circulating levels of sPLA₂ were found to provide an accurate prognostic indicator for development of CAD in healthy individuals (Mallat 2007). Measurement of sPLA₂ activity has been shown to be an independent predictor of death and new or recurrent MI in subjects with ACS, and provides greater prognostic accuracy than measuring type IIA concentration only (Mallat 2005). It has also been proposed that sPLA₂ may have detrimental effects in the setting of ischemic events. This is based largely on the finding of sPLA₂ depositions in the necrotic center of infarcted human myocardium (Nijmeijer 2002).

Previous studies have established that once- or twice-daily administration of the sPLA₂ inhibitor Amino-1,2-dioxoethyl)-2-ethyl-1-(phenylmethyl)-1H-indol-4-yl]oxy]acetic acid methyl ester (A-002) decreases total cholesterol, LDL-C, total LDL-C particle, and small LDL-C particle levels in a stable CVD population, as well as in diabetic and high baseline LDL-C subpopulations (WO2008/137803). In addition, these previous studies established that administration of A-002 in combination with one or more statins results in a synergistic decrease in LDL-C and small LDL-C particle levels in a stable CVD population, including in a high baseline LDL level subpopulation. This effect was not limited to a particular statin, but instead was observed across the entire spectrum of statins. Previous studies have also established that A-002 administration decreases levels of various inflammatory markers such as hs-CRP and sPLA₂ in stable populations.

These previous findings support the use of sPLA₂ inhibitors alone or in combination with other cardiovascular drugs to alter cholesterol levels and treat CVD in a stable population, including subjects with chronically elevated cholesterol levels. However, the ability to lower cholesterol levels and treat CVD in a stable population does not necessarily correlate with the ability to rapidly lower cholesterol levels and reduce MACEs in an unstable population that has recently experienced an ACS event. As discussed above, the acute inflammatory response following an ACS event places these unstable subjects at very high risk of MACEs. For this reason, therapeutics that successfully lower cholesterol levels and decrease MACEs in stable populations have proven to be less successful in unstable ACS populations. For example, one study examining the effect of 80 mg atorvastatin administration in subjects that had recently experienced an ACS event found only a 2.6% absolute reduction and a 16% relative reduction in death, non-fatal MI, cardiac arrest with resuscitation, or worsening symptomatic myocardial ischemia (Schwartz 2001). Given the high baseline levels of inflammation in diabetic subjects even before an ACS event occurs, the instability associated with the acute inflammatory response following an ACS event is especially pronounced. This makes standard CVD therapeutics even less successful at preventing MACEs following an ACS event in this population.

As disclosed in U.S. patent application Ser. No. 12/642,692, administration of A-002 in combination with statins to unstable patients that have recently experienced an ACS event reduces inflammation (as evidenced by decreases in mean and median levels of the inflammatory markers hs-CRP, sPLA₂, and IL-6), LDL levels, and MACE occurrence. Data from this clinical trial was analyzed to determine whether these effects could be observed in a diabetic subpopulation. As disclosed herein, administration of A-002 in combination with statin was found to reduce hs-CRP and IL-6 levels and the occurrence of UA requiring urgent hospitalization in diabetic subjects during the 16 weeks following ACS event occurrence and/or diagnosis. This is important because it establishes that A-002 in combination with statin is capable of decreasing at least one type of MACE in a population that is particularly vulnerable to cardiovascular disease due to high baseline inflammation levels.

Based on the results disclosed herein, methods are provided for treating MACEs, including reducing the likelihood of MACEs, and inhibiting inflammation in a diabetic subject who has previously experienced an ACS event by administering a therapeutically effective amount of one or more PLA₂ inhibitors alone or in combination with one or more therapeutics used in the treatment of MACEs or ACS. In certain embodiments, the one or more PLA₂ inhibitors are selected from sPLA₂, Lp-PLA₂, and cPLA₂ inhibitors, and in certain of these embodiments one or more of the PLA₂ inhibitors are sPLA₂ inhibitors. In certain embodiments, the one or more therapeutics used in the treatment of MACEs or ACS include one or more statins. Further provided herein are compositions, products, pharmaceutical formulations, and kits comprising one or more PLA₂ inhibitors alone or in combination with one or more therapeutics used in the treatment of MACEs or ACS for use in treating MACEs and/or inhibiting inflammation in post-ACS event diabetic subjects, as well as the use of one or more PLA₂ inhibitors alone or in combination with one or more MACE or ACS therapeutics to create a medicament for use in the methods disclosed herein.

In certain embodiments, an sPLA₂ inhibitor for use in the methods and compositions disclosed herein may be an indole-based sPLA₂ inhibitor, meaning that the compound contains an indole nucleus having the structure:

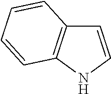

A variety of indole-based sPLA₂ inhibitors are known in the art. For example, indole-based sPLA₂ inhibitors that may be used in conjunction with the present invention include but are not limited to those set forth in U.S. Pat. Nos. 5,654,326 (Bach); 5,733,923 (Bach); 5,919,810 (Bach); 5,919,943 (Bach); 6,175,021 (Bach); 6,177,440 (Bach); 6,274,578 (Denney); and 6,433,001 (Bach), the entire disclosures of which are incorporated by reference herein. Methods of making indole-based sPLA₂ inhibitors are set forth in, for example, U.S. Pat. Nos. 5,986,106 (Khau); 6,265,591 (Anderson); and 6,380,397 (Anderson), the entire disclosures of which are incorporated by reference herein. sPLA₂ inhibitors for use in the present invention may be generated using these synthesis methods, or using any other synthesis method known in the art. In certain embodiments, sPLA₂ inhibitors for use in the present invention may be sPLA₂ type IIA, type V, and/or type X inhibitors. Various examples of indole-based sPLA₂ inhibitors are set forth below. These examples are merely provided as illustrations of the types of inhibitors that may be used in conjunction with the methods and compositions disclosed herein, and as such are not meant to be limiting. One of ordinary skill in the art will recognize that a variety of other indole-based sPLA₂ inhibitors may be used.

In certain embodiments, sPLA₂ inhibitors for use in the current invention are 1H-indole-3-glyoxylamide compounds having the structure:

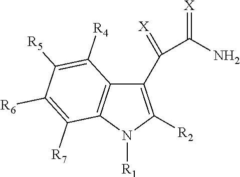

wherein:
each X is independently oxygen or sulfur;
R₁ is selected from the group consisting of (a), (b), and (c), wherein:

(a) is C₇-C₂₀ alkyl, C₇-C₂₀ alkenyl, C₇-C₂₀ alkynyl, carbocyclic radicals, or heterocyclic radicals;

(b) is a member of (a) substituted with one or more independently selected non-interfering substituents; and

(c) is the group -(L)-R₈₀, where, -(L)- is a divalent linking group of 1 to 12 atoms selected from carbon, hydrogen, oxygen, nitrogen, and sulfur, wherein the combination of atoms in -(L)- are selected from the group consisting of (i) carbon and hydrogen only, (ii) sulfur only, (iii) oxygen only, (iv) nitrogen and hydrogen only, (v) carbon, hydrogen, and sulfur only, and (vi) carbon, hydrogen, and oxygen only; and where R₈₀ is a group selected from (a) or (b); R₂ is hydrogen, halo, C₁-C₃ alkyl, C₃-C₄ cycloalkyl, C₃-C₄ cycloalkenyl, —O—(C₁-C₂ alkyl), —S—(C₁-C₂ alkyl), or a non-interfering substituent having a total of 1 to 3 atoms other than hydrogen; R₄ and R₅ are independently selected from the group consisting of hydrogen, a non-interfering substituent, and -(Lₐ)-(acidic group), wherein -(Lₐ)- is an acid linker having an acid linker length of 1 to 4; provided that at least one of R₄ and R₅ must be -(Lₐ)-(acidic group); R₆ and R₇ are each independently selected from hydrogen, non-interfering substituents, carbocyclic radicals, carbocyclic radicals substituted with non-interfering substituents, heterocyclic radicals, and heterocyclic radicals substituted with non-interfering substituents; provided that for any of the groups R₁, R₆, and R₇, the carbocyclic radical is selected from the group consisting of cycloalkyl, cycloalkenyl, phenyl, naphthyl, norbornanyl, bicycloheptadienyl, toluoyl, xylenyl, indenyl, stilbenzyl, terphenylyl, diphenylethylenyl, phenyl-cyclohexenyl, acenaphthylenyl, and anthracenyl, biphenyl, bibenzylyl and related bibenzylyl homologues represented by the formula (bb),

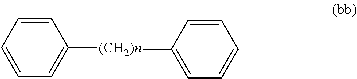

where n is a number from 1 to 8; provided, that for any of the groups R₁, R₆, and R₇, the heterocyclic radical is selected from the group consisting of pyrrolyl, furanyl, thiophenyl, pyrazolyl, imidazolyl, phenylimidazolyl, triazolyl, isoxazolyl, oxazolyl, thiazolyl, thiadiazolyl, indolyl, carbazolyl, norharmanyl, azaindolyl, benzofuranyl, dibenzofuranyl, thianaphtheneyl, dibenzothiophenyl, indazolyl, imidazo(1.2-A)pyridinyl, benzotriazolyl, anthranilyl, 1,2-benzisoxazolyl, benzoxazolyl, benzotriazolyl, purinyl, pyridinyl, dipyridylyl. phenylpyridinyl, benzylpyridinyl, pyrimidinyl, phenylpyrimidinyl, pyrazinyl, 1,3,5-triazinyl, quinolinyl, phthalazinyl, quinazolinyl, and quinoxalinyl; and
provided that for the groups R₁, R₂, R₄, R₅, R₆, and R₇ the non-interfering substituent is selected from the group consisting of C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, toluoyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl, where n is from 1 to 8;
and pharmaceutically acceptable salts, solvates, prodrug derivatives, racemates, tautomers, or optical isomers thereof.

In certain of these embodiments, -(L)- has the formula:

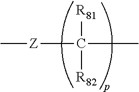

wherein R₈₁ and R₈₂ are each independently selected from the group consisting of hydrogen, C₁-C₁₀ alkyl, carboxy, carbalkoxy, and halo; p is a number from 1 to 5; and Z is selected from the group consisting of a bond, —(CH₂)—, —O—, —N(C₁-C₁₀ alkyl)-, —NH—, and —S—.

In certain of these embodiments wherein R₄ is -(Lₐ)—(acidic group), the acid linker -(Lₐ)- has the formula:

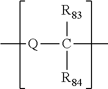

wherein Q is selected from the group consisting of —(CH₂)—, —O—, —NH—, and —S—; and R₈₃ and R₈₄ are each independently selected from the group consisting of hydrogen, C₁-C₁₀ alkyl, aryl, C₁-C₁₀ alkaryl, C₁-C₁₀ aralkyl, hydroxy, and halo.

In certain of these embodiments wherein R₅ is -(Lₐ)-(acidic group), the acid linker -(Lₐ)- has the formula:

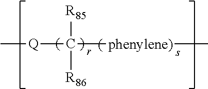

wherein r is a number from 2 to 7; s is 0 or 1; Q is selected from the group consisting of —(CH₂)—, —O—, —NH—, and —S—; and R₈₅ and R₈₆ are each independently selected from the group consisting of hydrogen, C₁-C₁₀ alkyl, aryl, C₁-C₁₀ alkaryl, C₁-C₁₀ aralkyl, carboxy, carbalkoxy, and halo.

In certain embodiments, a 1H-indole-3-glyoxylamide compound for use in the present invention is selected from the group consisting of: ((3-(2-Amino-1,2-dioxoethyl)-2-ethyl-1-(phenylmethyl)-1H-indol-4-yl)oxy)acetic acid; [[3-(2-Amino-1,2-dioxoethyl)-2-ethyl-1-(phenylmethyl)-1H-indol-4-yl]oxy]acetic acid methyl ester; ((3-(2-Amino-1,2-dioxoethyl)-2-methyl-1-(phenylmethyl)-1H-indol-4-yl)oxy)acetic acid; dl-2-((3-(2-Amino-1,2-dioxoethyl)-2-methyl-1-(phenylmethyl)-1H-indol-4-yl)oxy)propanoic acid; ((3-(2-Amino-1,2-dioxoethyl)-1-((1,1′-biphenyl)-2-ylmethyl)-2-methyl-1H-indol-4-yl)oxy)acetic acid; ((3-(2-Amino-1,2-dioxoethyl)-1-((1,1′-biphenyl)-3-ylmethyl)-2-methyl-1H-indol-4-yl)oxy)acetic acid; ((3-(2-Amino-1,2-dioxoethyl)-1-((1,1′-biphenyl)-4-ylmethyl)-2-methyl-1H-indol-4-yl)oxy)acetic acid; ((3-(2-Amino-1,2-dioxoethyl)-1-((2,6-dichlorophenyl)methyl)-2-methyl-1H-indol-4-yl)oxy)acetic acid; ((3-(2-Amino-1,2-dioxoethyl)-1-(4(-fluorophenyl)methyl)-2-methyl-1H-indol-4-yl)oxy)acetic acid; ((3-(2-Amino-1,2-dioxoethyl)-2-methyl-1-((1-naphthalenyl)methyl)-1H-indol-4-yl)oxy)acetic acid; ((3-(2-Amino-1,2-dioxoethyl)-1-((3-chlorophenyl)methyl)-2-ethyl-1H-indol-4-yl)oxy)acetic acid; ((3-(2-Amino-1,2-dioxoethyl)-1-((1,1′-biphenyl)-2-ylmethyl)-2-ethyl-1H-indol-4-yl)oxy)acetic acid; ((3-(2-amino-1,2-dioxoethyl)-1-((1,1′-biphenyl)-2-ylmethyl)-2-propyl-1H-indol-4-yl)oxy)acetic acid; ((3-(2-Amino-1,2-dioxoethyl)-2-cyclopropyl-1-(phenylmethyl)-1H-indol-4-yl)oxy)acetic acid; ((3-(2-Amino-1,2-dioxoethyl)-1-((1,1′-biphenyl)-2-ylmethyl)-2-cyclopropyl-1H-indol-4-yl)oxy)acetic acid; and 4-43-(2-Amino-1,2-dioxoethyl)-2-ethyl-1-(phenylmethyl)-1H-indol-5-yl)oxy)butanoic acid, or pharmaceutically acceptable salts, solvates, prodrug derivatives, racemates, tautomers, or optical isomers thereof.

In certain embodiments, sPLA₂ inhibitors for use in the current invention are 1H-indole-3-glyoxylamide compounds having the structure:

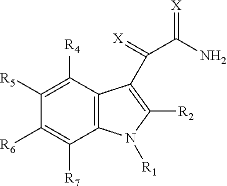

wherein:
both X are oxygen;
R₁ is selected from the group consisting of:

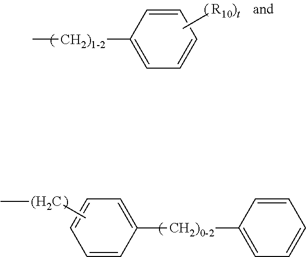

wherein R₁₀ is a radical independently selected from halo, C₁-C₁₀ alkoxy, —S—(C₁-C₁₀ alkyl), and C₁-C₁₀ haloalkyl, and t is a number from 0 to 5;
R₂ is selected from the group consisting of halo, cyclopropyl, methyl, ethyl, and propyl;
R₄ and R₅ are independently, selected from the group consisting of hydrogen, a non-interfering substituent, and -(Lₐ)-(acidic group), wherein -(Lₐ)- is an acid linker; provided that the acid linker -(Lₐ)- for R₄ is selected from the group consisting of:

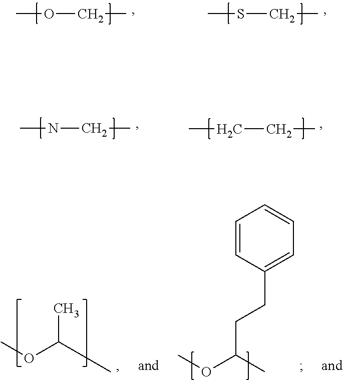

provided that the acid linker -(Lₐ)- for R₅ is selected from the group consisting of:

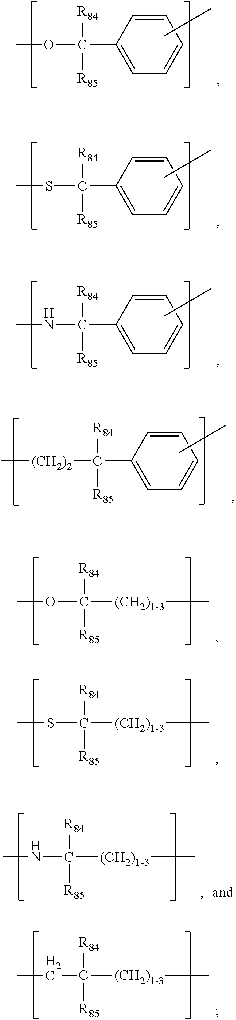

wherein R₈₄ and R₈₅ are each independently selected from the group consisting of hydrogen, C₁-C₁₀ alkyl, aryl, C₁-C₁₀ alkaryl, C₁-C₁₀ aralkyl, carboxy, carbalkoxy, and halo; provided that at least one of R₄ and R₅ must be -(Lₐ)-(acidic group), and (acidic group) on -(Lₐ)-(acidic group) of R₄ or R₅ is selected from —CO₂H, —SO₃H, or —P(O)(OH)₂;
R₆ and R₇ are each independently selected from the group consisting of hydrogen and non-interfering substituents, with the non-interfering substituents being selected from the group consisting of: C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, toluoyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; wherein n is from 1 to 8;
and pharmaceutically acceptable salts, solvates, prodrug derivatives, racemates, tautomers, or optical isomers thereof.

In certain embodiments, 1H-indole-3-glyoxylamide compounds for use in the present invention are selected from the group consisting of: ((3-(2-Amino-1,2-dioxoethyl)-2-methyl-1-(phenylmethyl)-1H-indol-4-yl)oxy)acetic acid; ((3-(2-Amino-1,2-dioxoethyl)-2-methyl-1-(phenylmethyl)-1H-indol-4-yl)oxy)acetic acid methyl ester; dl-2-((3-(2-Amino-1,2-dioxoethyl)-2-methyl-1-(phenylmethyl)-1H-indol-4-yl)oxy)propanoic acid; dl-2-((3-(2-Amino-1,2-dioxoethyl)-2-methyl-1-(phenylmethyl)-1H-indol-4-yl)oxy)propanoic acid methyl ester; ((3-(2-Amino-1,2-dioxoethyl)-1-((1,1′-biphenyl)-2-ylmethyl)-2-methyl-1H-indol-4-yl)oxy)acetic acid; ((3-(2-Amino-1,2-dioxoethyl)-1-((1,1′-biphenyl)-2-ylmethyl)-2-methyl-1H-indol-4-yl)oxy)acetic acid methyl ester; ((3-(2-Amino-1,2-dioxoethyl)-1-((1,1′-biphenyl)-3-ylmethyl)-2-methyl-1H-indol-4-yl)oxy)acetic acid; ((3-(2-Amino-1,2-dioxoethyl)-1-((1,1′-biphenyl)-3-ylmethyl)-2-methyl-1H-indol-4-yl)oxy)acetic acid methyl ester; ((3-(2-Amino-1,2-dioxoethyl)-1-((1,1′-biphenyl)-4-ylmethyl)-2-methyl-1H-indol-4-yl)oxy)acetic acid; ((3-(2-Amino-1,2-dioxoethyl)-1-((1,1′-biphenyl)-4-ylmethyl)-2-methyl-1H-indol-4-yl)oxy)acetic acid methyl ester; ((3-(2-Amino-1,2-dioxoethyl)-1-((2,6-dichlorophenyl)methyl)-2-methyl-1H-indol-4-yl)oxy)acetic acid; ((3-(2-Amino-1,2-dioxoethyl)-1-((2,6-dichlorophenyl)methyl)-2-methyl-1H-indol-4-yl)oxy)acetic acid methyl ester; ((3-(2-Amino-1,2-dioxoethyl)-1-(4(-fluorophenyl)methyl)-2-methyl-1H-indol-4-yl)oxy)acetic acid; ((3-(2-Amino-1,2-dioxoethyl)-1-(4(-fluorophenyl)methyl)-2-methyl-1H-indol-4-yl)oxy)acetic acid methyl ester; ((3-(2-Amino-1,2-dioxoethyl)-2-methyl-1-((1-naphthalenyl)methyl)-1H-indol-4-yl)oxy)acetic acid; ((3-(2-Amino-1,2-dioxoethyl)-2-methyl-1-((1-naphthalenyl)methyl)-1H-indol-4-yl)oxy)acetic acid methyl ester; ((3-(2-Amino-1,2-dioxoethyl)-1-((3-chlorophenyl)methyl)-2-ethyl-1H-indol-4-yl)oxy)acetic acid; ((3-(2-Amino-1,2-dioxoethyl)-1-((3-chlorophenyl)methyl)-2-ethyl-1H-indol-4-yl)oxy)acetic acid methyl ester; ((3-(2-Amino-1,2-dioxoethyl)-1-((1,1′-biphenyl)-2-ylmethyl)-2-ethyl-1H-indol-4-yl)oxy)acetic acid; ((3-(2-Amino-1,2-dioxoethyl)-1-((1,1′-biphenyl)-2-ylmethyl)-2-ethyl-1H-indol-4-yl)oxy)acetic acid methyl ester; ((3-(2-amino-1,2-dioxoethyl)-1-((1,1′-biphenyl)-2-ylmethyl)-2-propyl-1H-indol-4-yl)oxy)acetic acid; ((3-(2-amino-1,2-dioxoethyl)-1-((1,1′-biphenyl)-2-ylmethyl)-2-propyl-1H-indol-4-yl)oxy)acetic acid methyl ester; ((3-(2-Amino-1,2-dioxoethyl)-2-cyclopropyl-1-(phenylmethyl)-1H-indol-4-yl)oxy)acetic acid; ((3-(2-Amino-1,2-dioxoethyl)-2-cyclopropyl-1-(phenylmethyl)-1H-indol-4-yl)oxy)acetic acid methyl ester; ((3-(2-Amino-1,2-dioxoethyl)-1-((1,1′-biphenyl)-2-ylmethyl)-2-cyclopropyl-1H-indol-4-yl)oxy)acetic acid; ((3-(2-Amino-1,2-dioxoethyl)-14(1,1′-biphenyl)-2-ylmethyl)-2-cyclopropyl-1H-indol-4-yl)oxy)acetic acid methyl ester; 4-((3-(2-Amino-1,2-dioxoethyl)-2-ethyl-1-(phenylmethyl)-1H-indol-5-yl)oxy)butanoic acid; 4-((3-(2-Amino-1,2-dioxoethyl)-2-ethyl-1-(phenylmethyl)-1H-indol-5-yl)oxy)butanoic acid tert-butyl ester, or pharmaceutically acceptable salts, solvates, prodrug derivatives, racemates, tautomers, or optical isomers thereof.

In certain embodiments, sPLA₂ inhibitors for use in the current invention are 1H-indole-3-glyoxylamide compounds having the structure:

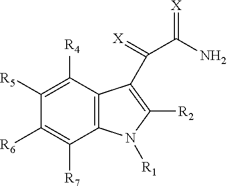

wherein:
each X is independently oxygen or sulfur;
R₁ is selected from groups (a), (b), and (c) wherein:

(a) is C₇-C₂₀ alkyl, C₇-C₂₀ alkenyl, C₇-C₂₀ alkynyl, carbocyclic radical, or heterocyclic radical;

(b) is a member of (a) substituted with one or more independently selected non-interfering substituents; and

(c) is the group -(L)-R₈₀, wherein -(L)- is a divalent linking group of 1 to 12 atoms selected from carbon, hydrogen, oxygen, nitrogen, and sulfur; wherein the combination of atoms in -(L)- are selected from the group consisting of (i) carbon and hydrogen only, (ii) sulfur only, (iii) oxygen only, (iv) nitrogen and hydrogen only, (v) carbon, hydrogen, and sulfur only, and (vi) and carbon, hydrogen, and oxygen only; and where R₈₀ is a group selected from (a) or (b); R₂ is selected from the group consisting of hydrogen, halo, C₁-C₃ alkyl, C₃-C₄ cycloalkyl, C₃-C₄ cycloalkenyl, —O—(C₁-C₂ alkyl), —S—(C₁-C₂ alkyl), and a non-interfering substituent having a total of 1 to 3 atoms other than hydrogen;

R₄ and R₅ are independently selected from the group consisting of hydrogen, a non-interfering substituent, and the group -(Lₐ)-(acidic group), wherein -(Lₐ)- is an acid linker having an acid linker length of 1 to 4; provided that at least one of R₄ and R₅ is -(Lₐ)-(acidic group);
R₆ and R₇ are each independently selected from the group consisting of hydrogen, non-interfering substituents, carbocyclic radicals, carbocyclic radicals substituted with non-interfering substituents, heterocyclic radicals, and heterocyclic radicals substituted with non-interfering substituents;
and pharmaceutically acceptable salts, solvates, prodrug derivatives, racemates, tautomers, or optical isomers thereof.

In certain embodiments, sPLA₂ inhibitors for use in the current invention are methyl ester prodrug derivatives of 1H-indole-3-glyoxylamide compounds having the structure:

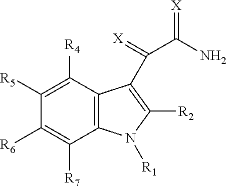

wherein:
both X are oxygen;
R₁ is selected from the group consisting of:

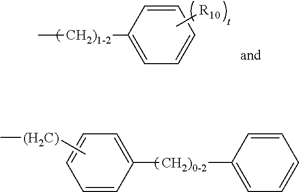

wherein R₁₀ is a radical independently selected from halo, C₁-C₁₀ alkyl, C₁-C₁₀ alkoxy, —S—(C₁-C₁₀ alkyl), and C₁-C₁₀ haloalkyl, and t is a number from 0 to 5;
R₂ is selected from the group consisting of halo, cyclopropyl, methyl, ethyl, and propyl;
R₄ and R₅ are independently selected from the group consisting of hydrogen, a non-interfering substituent, and -(Lₐ)-(acidic group), wherein -(Lₐ)- is an acid linker; provided that the acid linker -(Lₐ)- for R₄ is selected from the group consisting of:

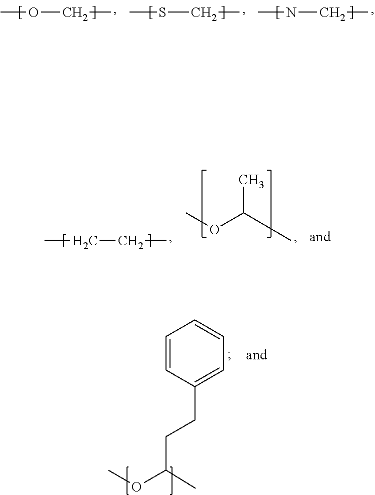

provided that the acid linker -(Lₐ)- for R₅ is selected from the group consisting of:

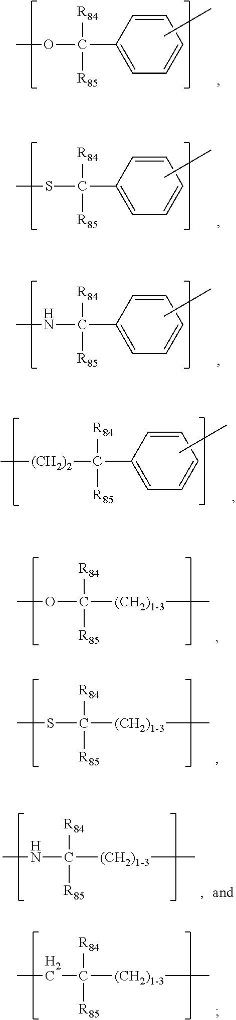

wherein R₈₄ and R₈₅ are each independently selected from the group consisting of hydrogen, C₁-C₁₀ alkyl, aryl, C₁-C₁₀ alkaryl, C₁-C₁₀ aralkyl, carboxy, carbalkoxy, and halo; provided that at least one of R₄ and R₅ must be -(Lₐ)-(acidic group), and (acidic group) on -(Lₐ)-(acidic group) of R₄ or R₅ is selected from —CO₂H, —SO₃H, or —P(O)(OH)₂;
R₆ and R₇ are each independently selected from the group consisting of hydrogen and non-interfering substituents, with the non-interfering substituents being selected from the group consisting of: C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, toluoyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; wherein n is from 1 to 8;
and pharmaceutically acceptable salts, solvates, prodrug derivatives, racemates, tautomers, or optical isomers thereof.

In certain embodiments, sPLA₂ inhibitors for use in the current invention are (acyloxy) alkyl ester prodrug derivatives of 1H-indole-3-glyoxylamide compounds having the structure:

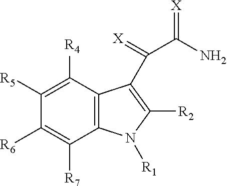

wherein:
both X are oxygen;
R₁ is selected from the group consisting of:

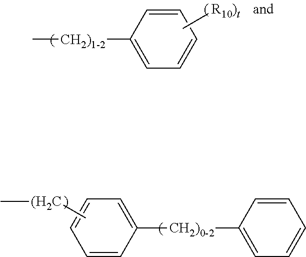

wherein R₁₀ is a radical independently selected from halo, C₁-C₁₀ alkyl, C₁-C₁₀ alkoxy, —S—(C₁-C₁₀ alkyl), and C₁-C₁₀ haloalkyl, and t is a number from 0 to 5;
R₂ is selected from the group consisting of halo, cyclopropyl, methyl, ethyl, and propyl;
R₄ and R₅ are independently selected from the group consisting of hydrogen, a non-interfering substituent, and -(Lₐ)-(acidic group), wherein -(Lₐ)- is an acid linker; provided that the acid linker -(Lₐ)- for R₄ is selected from the group consisting of:

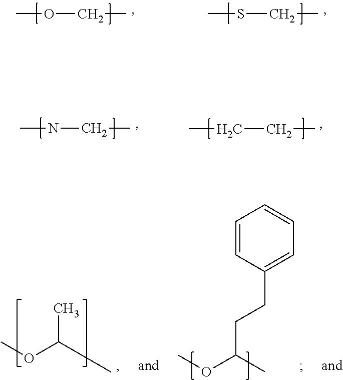

provided that the acid linker -(Lₐ)- for R₅ is selected from the group consisting of:

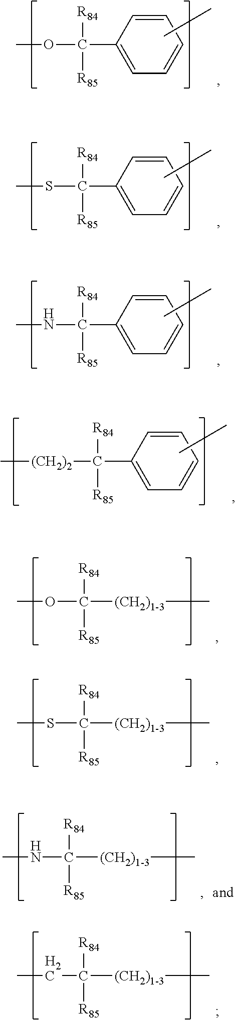

wherein R₈₄ and R₈₅ are each independently selected from the group consisting of hydrogen, C₁-C₁₀ alkyl, aryl, C₁-C₁₀ alkaryl, C₁-C₁₀ aralkyl, carboxy, carbalkoxy, and halo; provided that at least one of R₄ and R₅ must be -(Lₐ)-(acidic group), and (acidic group) on -(Lₐ)-(acidic group) of R₄ or R₅ is selected from —CO₂H, —SO₃H, or —P(O)(OH)₂;
R₆ and R₇ are each independently selected from the group consisting of hydrogen and non-interfering substituents, with the non-interfering substituents being selected from the group consisting of: C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, toluoyl, xylenyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —(CONHSO₂R), —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; wherein n is from 1 to 8;
and pharmaceutically acceptable salts, solvates, prodrug derivatives, racemates, tautomers, or optical isomers thereof.

In certain embodiments, sPLA₂ inhibitors for use in the current invention are substituted tricyclics having the structure:

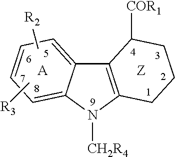

wherein:
R₁ is selected from the group consisting of —NHNH₂ and —NH₂;
R₂ is selected from the group consisting of —OH and —O(CH₂)ₘR₅; wherein R₅ is selected from the group consisting of H, —CO₂H, —CO₂(C₁-C₄ alkyl), —SO₃H, —SO₃(C₁-C₄ alkyl), tetrazolyl, —CN, —NH₂, —NHSO₂R₁₅, —CONHSO₂R₁₅, phenyl, phenyl substituted with —CO₂H or —CO₂(C₁-C₄)alkyl, and

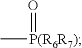

wherein R₆ and R₇ are each independently selected from the group consisting of —OH, —O(C₁-C₄)alkyl; R₁₅ is selected from the group consisting of —(C₁-C₆)alkyl and —CF₃; and m is 1-3;
R₃ is selected from the group consisting of H, —O(C₁-C₄)alkyl, halo, —(C₁-C₆)alkyl, phenyl, —(C₁-C₄)alkylphenyl, phenyl substituted with —(C₁-C₆)alkyl, halo, or —CF₃, —CH₂OSi(C₁-C₆)alkyl, furyl, thiophenyl, —(C₁-C₆)hydroxyalkyl, and —(CH₂)ₙR₈; wherein R₈ is selected from the group consisting of H, —CONH₂, —NR₉R₁₀, —CN, and phenyl; wherein R₉ and R₁₀ are each independently —(C₁-C₄)alkyl or -phenyl(C₁-C₄)alkyl; and n is 1 to 8;
R₄ is selected from the group consisting of H, —(C₅-C₁₄)alkyl, —(C₃-C₁₄)cycloalkyl, pyridyl, phenyl, and phenyl substituted with —(C₁-C₆)alkyl, halo, —CF₃, —OCF₃, —(C₁-C₄)alkoxy, —CN, —(C₁-C₄)alkylthio, phenyl(C₁-C₄)alkyl, —(C₁-C₄)alkylphenyl, phenyl, phenoxy, or naphthyl;
A is selected from the group consisting of phenyl and pyridyl wherein the nitrogen is at the 5-, 6-, 7-, or 8-position;
Z is selected from the group consisting of cyclohexenyl, phenyl, pyridyl wherein the nitrogen is at the 1-, 2-, or 3-position, and a 6-membered heterocyclic ring having one heteroatom selected from the group consisting of sulfur and oxygen at the 1-, 2-, or 3-position and nitrogen at the 1-, 2-, 3-, or 4- position, or wherein one carbon on the heterocyclic ring is optionally substituted with ═O; and wherein one of A or Z is a heterocyclic ring;
and pharmaceutically acceptable salts, solvates, prodrug derivatives, racemates, tautomers, or optical isomers thereof.

In certain embodiments, sPLA₂ inhibitors for use in the current invention are substituted tricyclics having the structure:

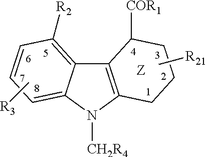

wherein:
Z is selected from the group consisting of cyclohexenyl and phenyl;
R₂₁ is a non-interfering substituent;

### R₁ is —NHNH₂ or —NH₂;

R₂ is selected from the group consisting of —OH and —O(CH₂)ₘR₅; wherein R₅ is selected from the group consisting of H, —CO₂H, —CONH₂, —CO₂(C₁-C₄ alkyl), —SO₃H, —SO₃(C₁-C₄ alkyl), tetrazolyl, —CN, —NH₂, —NHSO₂R₁₅, —CONHSO₂R₁₅, phenyl, phenyl substituted with —CO₂H or —CO₂(C₁-C₄)alkyl, and

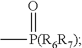

wherein R₆ and R₇ are each independently selected from the group consisting of —OH, —O(C₁-C₄)alkyl; R₁₅ is selected from the group consisting of —(C₁-C₆)alkyl and —CF₃; and m is 1-3;
R₃ selected from the group consisting of H, —O(C₁-C₄)alkyl, halo, —(C₁-C₆)alkyl, phenyl, —(C₁-C₄)alkylphenyl, phenyl substituted with —(C₁-C₆)alkyl, halo, or —CF₃, —CH₂OSi(C₁-C₆)alkyl, furyl, thiophenyl, —(C₁-C₆)hydroxyalkyl, and —(CH₂)ₙR₈; wherein R₈ is selected from the group consisting of H, —CONH₂, —NR₉R₁₀, —CN, and phenyl; R₉ and R₁₀ are each independently selected from the group consisting of H, —CF₃, phenyl, —(C₁-C₄)alkyl, —(C₁-C₄)alkylphenyl, and -phenyl(C₁-C₄)alkyl; and n is 1 to 8;
R₄ is selected from the group consisting of H, —(C₅-C₁₄)alkyl, —(C₃-C₁₄)cycloalkyl, pyridyl, phenyl, phenyl substituted with —(C₁-C₆)alkyl, halo, —CF₃, —OCF₃, —(C₁-C₄)alkoxy, —CN, —(C₁-C₄)alkylthio, -phenyl(C₁-C₄)alkyl, —(C₁-C₄)alkylphenyl, phenyl, phenoxy and naphthyl;
and pharmaceutically acceptable salts, solvates, prodrug derivatives, racemates, tautomers, or optical isomers thereof.

In certain embodiments, sPLA₂ inhibitors for use in the current invention are selected from the group consisting of: {9-[phenyl)methyl]-5-carbamoylcarbazol-4-yl}oxyacetic acid; 9-benzyl-5,7-dimethoxy-1,2,3,4-tetrahydrocarbazole-4-carboxylic acid hydrazide; 9-benzyl-5,7-dimethoxy-1,2,3,4-tetrahydrocarbazole-4-carboxamide; [9-benzyl-4-carbamoyl-7-methoxy-1,2,3,4-tetrahydrocarbazol-5-yl]oxyacetic acid; [9-benzyl-4-carbamoyl-7-methoxycarbazol-5-yl]oxyacetic acid; methyl [9-benzyl-4-carbamoyl-7-methoxycarbazol-5-yl]oxyacetic acid; 9-benzyl-7-methoxy-5-cyanomethyloxy-1,2,3,4-tetrahydrocarbazole-4-carboxamide; 9-benzyl-7-methoxy-5-(1H-tetrazol-5-yl-methyl)oxy)-1,2,3,4-tetrahydrocarbazole-4-carboxamide; {9-[phenyl)methyl]-5-carbamoyl-2-methyl-carbazol-4-yl}oxyacetic acid; {9-[(3-fluorophenyl)methyl]-5-carbamoyl-2-methylcarbazol-4-yl}oxyacetic acid; {9-[3-methylphenyl)methyl]-5-carbamoyl-2-methylcarbazol-4-yl}oxyacetic acid; {9-[phenyl)methyl]-5-carbamoyl-2-(4-trifluoromethylphenyl)-carbazol-4-yl}oxyacetic acid; 9-benzyl-5-(2-methanesulfonamido)ethyloxy-7-methoxy-1,2,3,4-tetrahydrocarbazole-4-carboxamide; 9-benzyl-4-(2-methanesulfonamido)ethyloxy-2-methoxycarbazole-5-carboxamide; 9-benzyl-4-(2-trifluoromethanesulfonamido)ethyloxy-2-methoxycarbazole-5-carboxamide; 9-benzyl-5-methanesulfonamidoylmethyloxy-7-methoxy-1,2,3,4-tetrahydrocarbazole-4-carboxamide; 9-benzyl-4-methanesulfonamidoylmethyloxy-carbazole-5-carboxamide; [5-carbamoyl-2-pentyl-9-(phenylmethyl)carbazol-4-yl]oxyacetic acid; [5-carbamoyl-2-(1-methylethyl)-9-(phenylmethyl)carbazol-4-yl]oxyacetic acid; [5-carbamoyl-9-(phenylmethyl)-2-[tri(−1-methylethyl)silyl)oxymethyl]carbazol-4-yl]oxyacetic acid; [5-carbamoyl-2-phenyl-9-(phenylmethyl)carbazol-4-yl]oxyacetic acid; [5-carbamoyl-2-(4-chlorophenyl)-9-(phenylmethyl)carbazol-4-yl]oxyacetic acid; [5-carbamoyl-2-(2-furyl)-9-(phenylmethyl)carbazol-4-yl]oxyacetic acid; [5-carbamoyl-9-(phenylmethyl)-2-[tri(−1-methylethyl)silyl)oxymethyl]carbazol-4-yl]oxyacetic acid; {9-[(2-Fluorophenyl)methyl]-5-carbamoylcarbazol-4-yl}oxyacetic acid; {9-[(2-trifluoromethylphenyl)methyl]-5-carbamoylcarbazol-4-yl}oxyacetic acid; {9-[(2-benzylphenyl)methyl]-5-carbamoylcarbazol-4-yl}oxyacetic acid; {9-[(1-naphthyl)methyl]-5-carbamoylcarbazol-4-yl}oxyacetic acid; {9-[(2-cyanophenyl)methyl]-5-carbamoylcarbazol-4-yl}oxyacetic acid; {9-[(3-cyanophenyl)methyl]-5-carbamoylcarbazol-4-yl}oxyacetic acid; {9-[(3,5-dimethylphenyl)methyl]-5-carbamoylcarbazol-4-yl}oxyacetic acid; {9-[(3-iodophenyl)methyl]-5-carbamoylcarbazol-4-yl}oxyacetic acid; {9-[(2-Chlorophenyl)methyl]-5-carbamoylcarbazol-4-yl}oxyacetic acid; {9-[(2,3-difluorophenyl)methyl]-5-carbamoylcarbazol-4-yl}oxyacetic acid; {9-[(2,6-difluorophenyl)methyl]-5-carbamoylcarbazol-4-yl}oxyacetic acid; {9-[(2,6-dichlorophenyl)methyl]-5-carbamoylcarbazol-4-yl}oxyacetic acid; {9-[(2-biphenyl)methyl]-5-carbamoylcarbazol-4-yl}oxyacetic acid; {9-[(2-Biphenyl)methyl]-5-carbamoylcarbazol-4-yl}oxyacetic acid methyl ester; [9-Benzyl-4-carbamoyl-1,2,3,4-tetrahydrocarbazol-5-yl]oxyacetic acid; {9-[(2-Pyridyl)methyl]-5-carbamoylcarbazol-4-yl}oxyacetic acid; {9-[(3-Pyridyl)methyl]-5-carbamoylcarbazol-4-yl}oxyacetic acid; [9-benzyl-4-carbamoyl-8-methyl-1,2,3,4-tetrahydrocarbazol-5-yl]oxyacetic acid; [9-benzyl-5-carbamoyl-1-methylcarbazol-4-yl]oxyacetic acid; [9-benzyl-4-carbamoyl-8-fluoro-1,2,3,4-tetrahydrocarbazol-5-yl]oxyacetic acid; [9-benzyl-4-carbamoyl-8-chloro-1,2,3,4-tetrahydrocarbazol-5-yl]oxyacetic acid; [5-carbamoyl-9-(phenylmethyl)-2-[[(propen-3-yl)oxy]methyl]carbazol-4-yl]oxyacetic acid; [5-carbamoyl-9-(phenylmethyl)-2-[(propyloxy)methyl]carbazol-4-yl]oxyacetic acid; 9-benzyl-7-methoxy-5-((carboxamidomethyl)oxy)-1,2,3,4-tetrahydrocarbazole-4-carboxamide; 9-benzyl-7-methoxy-5-cyanomethyloxy-carbazole-4-carboxamide; 9-benzyl-7-methoxy-5-((1H-tetrazol-5-yl-methyl)oxy)-carbazole-4-carboxamide; 9-benzyl-7-methoxy-5-((carboxamidomethyl)oxy)-carbazole-4-carboxamide; [9-Benzyl-4-carbamoyl-1,2,3,4-tetrahydrocarbazole-5-yl]oxyacetic acid; {9-[(phenyl)methyl]-5-carbamoyl-2-methyl-carbazol-4-yl}oxyacetic acid; {9-[(3-fluorophenyl)methyl]-5-carbamoyl-2-methylcarbazol-4-yl}oxyacetic acid; {9-[(3-methylphenyl)methyl]-5-carbamoyl-2-methylcarbazol-4-yl}oxyacetic acid; {9-[(phenyl)methyl]-5-carbamoyl-2-(4-trifluoromethylphenyl)-carbazol-4-yl}oxyacetic acid; 9-benzyl-5-(2-methane sulfonamido)ethyloxy-7-methoxy-1,2,3,4-tetrahydrocarbazole-4-carboxamide; 9-benzyl-4-(2-methanesulfonamido)ethyloxy-2-methoxycarbazole-5-carboxamide; 9-benzyl-4-(2-trifluoromethanesulfonamido)ethyloxy-2-methoxycarbazole-5-carboxamide ; 9-benzyl-5-methanesulfonamidoylmethyloxy-7-methoxy-1,2,3,4-tetrahydrocarbazole-4-carboxamide; 9-benzyl-4-methanesulfonamidoylmethyloxy-carbazole-5-carboxamide; [5-carbamoyl-2-pentyl-9-(phenylmethyl)carbazol-4-yl]oxyacetic acid; [5-carbamoyl-2-(1-methylethyl)-9-(phenylmethyl)carbazol-4-yl]oxyacetic acid; [5-carbamoyl-9-(phenylmethyl)-2-[tri(−1-methylethyl)silyl)oxymethyl]carbazol-4-yl]oxyacetic acid; [5-carbamoyl-2-phenyl-9-(phenylmethyl)carbazol-4-yl]oxyacetic acid; [5-carbamoyl-2-(4-chlorophenyl)-9-(phenylmethyl)carbazol-4-yl]oxyacetic acid; [5-carbamoyl-2-(2-furyl)-9-(phenylmethyl)carbazol-4-yl]oxyacetic acid; [5-carbamoyl-9-(phenylmethyl)-2-[tri(−1-methylethyl)silyl)oxymethyl]carbazol-4-yl]oxyacetic acid; {9-[(3-fluorophenyl)methyl]-5-carbamoylcarbazol-4-yl}oxyacetic acid; {9-[(3-chlorophenyl)methyl]-5-carbamoylcarbazol-4-yl}oxyacetic acid; {9-[(3-phenoxyphenyl)methyl]-5-carbamoylcarbazol-4-yl}oxyacetic acid; {9-[(2-Fluorophenyl)methyl]-5-carbamoylcarbazol-4-yl}oxyacetic acid; {9-[(2-trifluoromethylphenyl)methyl]-5-carbamoylcarbazol-4-yl}oxyacetic acid; {9-[(2-benzylphenyl)methyl]-5-carbamoylcarbazol-4-yl}oxyacetic acid; {9-[(3-trifluoromethylphenyl)methyl]-5-carbamoylcarbazol-4-yl}oxyacetic acid; {9-[(1-naphthyl)methyl]-5-carbamoylcarbazol-4-yl}oxyacetic acid; {9-[(2-cyanophenyl)methyl]-5-carbamoylcarbazol-4-yl}oxyacetic acid; {9-[(3-cyanophenyl)methyl]-5-carbamoylcarbazol-4-yl}oxyacetic acid; {9-[(2-methylphenyl)methyl]-5-carbamoylcarbazol-4-yl}oxyacetic acid; {9-[(3-methylphenyl)methyl]-5-carbamoylcarbazol-4-yl}oxyacetic acid; {9-[(3,5-dimethylphenyl)methyl]-5-carbamoylcarbazol-4-yl}oxyacetic acid; {9-[(3-iodophenyl)methyl]-5-carbamoylcarbazol-4-yl}oxyacetic acid; {9-[(2-Chlorophenyl)methyl]-5-carbamoylcarbazol-4-yl}oxyacetic acid; {9-[(2,3-difluorophenyl)methyl]-5-carbamoylcarbazol-4-yl}oxyacetic acid; {9-[(2,6-difluorophenyl)methyl]-5-carbamoylcarbazol-4-yl}oxyacetic acid; {9-[(2,6-dichlorophenyl)methyl]-5-carbamoylcarbazol-4-yl}oxyacetic acid; {9-[(3-trifluoromethoxyphenyl)methyl]-5-carbamoylcarbazol-4-yl}oxyacetic acid; {9-[(2-biphenyl)methyl]-5-carbamoylcarbazol-4-yl}oxyacetic acid; {9-[(2-Biphenyl)methyl]-5-carbamoylcarbazol-4-yl}oxyacetic acid methyl ester; [9-Benzyl-4-carbamoyl-1,2,3,4-tetrahydrocarbazole-5-yl]oxyacetic acid; {9-[(2-Pyridyl)methyl]-5-carbamoylcarbazol-4-yl}oxyacetic acid; {9-[(3-Pyridyl)methyl]-5-carbamoylcarbazol-4-yl}oxyacetic acid; [9-benzyl-4-carbamoyl-8-methyl-1,2,3,4-tetrahydrocarbazol-5-yl]oxyacetic acid; [9-benzyl-5-carbamoyl-1-methylcarbazol-4-yl]oxyacetic acid; [9-benzyl-4-carbamoyl-8-fluoro-1,2,3,4-tetrahydrocarbazol-5-yl]oxyacetic acid; [9-benzyl-5-carbamoyl-1-fluorocarbazol-4-yl]oxyacetic acid; [9-benzyl-4-carbamoyl-8-chloro-1,2,3,4-tetrahydrocarbazol-5-yl]oxyacetic acid; [9-benzyl-5-carbamoyl-1-chlorocarbazol-4-yl]oxyacetic acid; [9-[(Cyclohexyl)methyl]-5-carbamoylcarbazol-4-yl]oxyacetic acid; [9-[(Cyclopentyl)methyl]-5-carbamoylcarbazol-4-yl]oxyacetic acid; [5-carbamoyl-9-(phenylmethyl)-2-(2-thienyl)carbazol-4-yl]oxyacetic acid; [5-carbamoyl-9-(phenylmethyl)-2-[[(propen-3-yl)oxy]methyl]carbazol-4-yl]oxyacetic acid; [5-carbamoyl-9-(phenylmethyl)-2-[(propyloxy)methyl]carbazol-4-yl]oxyacetic acid; 9-benzyl-7-methoxy-5-((carboxamidomethyl)oxy)-1,2,3,4-tetrahydrocarbazole-4-carboxamide; 9-benzyl-7-methoxy-5-cyanomethyloxy-carbazole-4-carboxamide; 9-benzyl-7-methoxy-5-((1H-tetrazol-5-yl-methyl)oxy)-carbazole-4-carboxamide; 9-benzyl-7-methoxy-5-((carboxamidomethyl)oxy)-carbazole-4-carboxamide; [9-Benzyl-4-carbamoyl-1,2,3,4-tetrahydrocarbazole-5-yl]oxyacetic acid; (R,S)-(9-benzyl-4-carbamoyl-1-oxo-3-thia-1,2,3,4-tetrahydrocarbazol-5-yl)oxyacetic acid; (R,S)-(9-benzyl-4-carbamoyl-3-thia-1,2,3,4-tetrahydrocarbazol-5-yl)oxyacetic acid; 2-(4-oxo-5-carboxamido-9-benzyl-9H-pyrido[3,4-b]indolyl)acetic acid chloride; [N-benzyl-1-carbamoyl-1-aza-1,2,3,4-tetrahydrocarbazol-8-yl]oxyacetic acid; 4-methoxy-6-methoxycarbonyl-10-phenylmethyl-6,7,8,9-tetrahydropyrido[1,2-a]indole; (4-carboxamido-9-phenylmethyl-4,5-dihydrothiopyrano[3,4-b]indol-5-yl)oxyacetic acid; 3,4-dihydro-4-carboxamidol-5-methoxy-9-phenylmethylpyrano[3,4-b]indole; 2-[(2,9 bis-benzyl-4-carbamoyl-1,2,3,4-tetrahydro-betacarbolin-5-yl)oxy]acetic acid; 2-[4-oxo-5-carboxamido-9-(2-methylbenzyl)-9H-pyrido[3,4-b]indolyl]acetic acid; 2-[4-oxo-5-carboxamido-9-(3-methylbenzyl)-9H-pyrido[3,4-b]indolyl]acetic acid; 2-[4-oxo-5-carboxamido-9-(4-methylbenzyl)-9H-pyrido[3,4-b]indolyl]acetic acid; 2-[4-oxo-5-carboxamido-9-(4-tert-butylbenzyl)-9H-pyrido[3,4-b]indolyl]acetic acid; 2-[4-oxo-5-carboxamido-9-pentafluorobenzyl-9H-pyrido[3,4-b]indolyl]acetic acid; 2-[4-oxo-5-carboxamido-9-(2-fluorobenzyl)-9H-pyrido[3,4-b]indolyl]acetic acid; 2-[4-oxo-5-carboxamido-9-(3-fluorobenzyl)-9H-pyrido[3,4-b]indolyl]acetic acid; 2-[4-oxo-5-carboxamido-9-(4-fluorobenzyl)-9H-pyrido[3,4-b]indolyl]acetic acid; 2-[4-oxo-5-carboxamido-9-(2,6-difluorobenzyl)-9H-pyrido[3,4-b]indolyl]acetic acid; 2-[4-oxo-5-carboxamido-9-(3,4-difluorobenzyl)-9H-pyrido[3,4-b]indolyl]acetic acid; 2-[4-oxo-5-carboxamido-9-(2,5-difluorobenzyl)-9H-pyrido[3,4-b]indolyl]acetic acid; 2-[4-oxo-5-carboxamido-9-(3,5-difluorobenzyl)-9H-pyrido[3,4-b]indolyl]acetic acid; 2-[4-oxo-5-carboxamido-9-(2,4-difluorobenzyl)-9H-pyrido[3,4-b]indolyl]acetic acid; 2-[4-oxo-5-carboxamido-9-(2,3-difluorobenzyl)-9H-pyrido[3,4-b]indolyl]acetic acid; 2-[4-oxo-5-carboxamido-9-[2-(trifluoromethyl)benzyl]-9H-pyrido[3,4-b]indolyl]acetic acid; 2-[4-oxo-5-carboxamido-9-[2-(trifluoromethyl)benzyl]-9H-pyrido[3,4-b]indolyl]acetic acid; 2-[4-oxo-5-carboxamido-9-[3-(trifluoromethyl)benzyl]-9H-pyrido[3,4-b]indolyl]acetic acid; 2-[4-oxo-5-carboxamido-9-[4-(trifluoromethyl)benzyl]-9H-pyrido[3,4-b]indolyl]acetic acid; 2-[4-oxo-5-carboxamido-9-[3,5-bis(trifluoromethyl)benzyl]-9H-pyrido[3,4-b]indolyl]acetic acid; 2-[4-oxo-5-carboxamido-9-[2,4-bis(trifluoromethyl)benzyl]-9H-pyrido[3,4-b]indolyl]acetic acid; 2-[4-oxo-5-carboxamido-9-(a-methylnaphthyl)-9H-pyrido[3,4-b]indolyl]acetic acid; 2-[4-oxo-5-carboxamido-9-(b-methylnaphthyl)-9H-pyrido[3,4-b]indolyl]acetic acid; 2-[4-oxo-5-carboxamido-9-(3,5-dimethylbenzyl)-9H-pyrido[3,4-b]indolyl]acetic acid; 2-[4-oxo-5-carboxamido-9-(2,4-dimethylbenzyl)-9H-pyrido[3,4-b]indolyl]acetic acid; 2-[4-oxo-5-carboxamido-9-(2-phenylbenzyl)-9H-pyrido[3,4-b]indolyl]acetic acid; 2-[4-oxo-5-carboxamido-9-(3-phenylbenzyl)-9H-pyrido[3,4-b]indolyl]acetic acid; 2-[4-oxo-5-carboxamido-9-(4-phenylbenzyl)-9H-pyrido[3,4-b]indolyl]acetic acid; 2-[4-oxo-5-carboxamido-9-(1-fluorenylmethy)-9H-pyrido[3,4-b]indolyl]acetic acid; 2-[4-oxo-5-carboxamido-9-(2-fluoro-3-methylbenzyl)-9H-pyrido[3,4-b]indolyl]acetic acid; 2-[4-oxo-5-carboxamido-9-(3-benzoylbenzyl)-9H-pyrido[3,4-b]indolyl]acetic acid; 2-[4-oxo-5-carboxamido-9-(2-phenoxybenzyl)-9H-pyrido[3,4-b]indolyl]acetic acid; 2-[4-oxo-5-carboxamido-9-(3-phenoxybenzyl)-9H-pyrido[3,4-b]indolyl]acetic acid; 2-[4-oxo-5-carboxamido-9-(4-phenoxybenzyl)-9H-pyrido[3,4-b]indolyl]acetic acid; 2-[4-oxo-5-carboxamido-9-[3-[2-(fluorophenoxy)benzyl]]-9H-pyrido[3,4-b]indolyl]acetic acid; 2-[4-oxo-5-carboxamido-9-[3-[4-(fluorophenoxy)benzyl]]-9H-pyrido[3,4-b]indolyl]acetic acid; 2-[4-oxo-5-carboxamido-9-[2-fluoro-3-(trifluoromethyl)benzyl]-9H-pyrido[3,4-b]indolyl]acetic acid; 2-[4-oxo-5-carboxamido-9-[2-fluoro-4-(trifluoromethyl)benzyl]-9H-pyrido[3,4-b]indolyl]acetic acid; 2-[4-oxo-5-carboxamido-9-[2-fluoro-5-(trifluoromethyl)benzyl]-9H-pyrido[3,4-b]indolyl]acetic acid; 2-[4-oxo-5-carboxamido-9-[3-fluoro-5-(trifluoromethyl)benzyl]-9H-pyrido[3,4-b]indolyl]acetic acid; 2-[4-oxo-5-carboxamido-9-[4-fluoro-2-(trifluoromethyl)benzyl]-9H-pyrido[3,4-b]indolyl]acetic acid; 2-[4-oxo-5-carboxamido-9-[4-fluoro-3-(trifluoromethyl)benzyl]-9H-pyrido[3,4-b]indolyl]acetic acid; 2-[4-oxo-5-carboxamido-9-[2-fluoro-6-(trifluoromethyl)benzyl]-9H-pyrido[3,4-b]indolyl]acetic acid; 2-[4-oxo-5-carboxamido-9-(2,3,6-trifluorobenzyl)-9H-pyrido[3,4-b]indolyl]acetic acid; 2-[4-oxo-5-carboxamido-9-(2,3,5-trifluorobenzyl)-9H-pyrido[3,4-b]indolyl]acetic acid; 2-[4-oxo-5-carboxamido-9-(2,4,5-trifluorobenzyl)-9H-pyrido[3,4-b]indolyl]acetic acid; 2-[4-oxo-5-carboxamido-9-(2,4,6-trifluorobenzyl)-9H-pyrido[3,4-b]indolyl]acetic acid; 2-[4-oxo-5-carboxamido-9-(2,3,4-trifluorobenzyl)-9H-pyrido[3,4-b]indolyl]acetic acid; 2-[4-oxo-5-carboxamido-9-(3,4,5-trifluorobenzyl)-9H-pyrido[3,4-b]indolyl]acetic acid; 2-[4-oxo-5-carboxamido-9-[3-(trifluoromethoxyl)benzyl]-9H-pyrido[3,4-b]indolyl]acetic acid; 2-[4-oxo-5-carboxamido-9-[4-(trifluoromethoxyl)benzyl]-9H-pyrido[3,4-b]indolyl]acetic acid; 2-[4-oxo-5-carboxamido-9-[4-methoxy(tetrafluoro)benzyl]-9H-pyrido[3,4-b]indolyl]acetic acid; 2-[4-oxo-5-carboxamido-9-(2-methoxybenzyl)-9H-pyrido[3,4-b]indolyl]acetic acid; 2-[4-oxo-5-carboxamido-9-(3-methoxybenzyl)-9H-pyrido[3,4-b]indolyl]acetic acid; 2-[4-oxo-5-carboxamido-9-(4-methoxybenzyl)-9H-pyrido[3,4-b]indolyl]acetic acid; 2-[4-oxo-5-carboxamido-9-(4-ethylbenzyl)-9H-pyrido[3,4-b]indolyl]acetic acid; 2-[4-oxo-5-carboxamido-9-(4-isopropylbenzyl)-9H-pyrido[3,4-b]indolyl]acetic acid; 2-[4-oxo-5-carboxamido-9-(3,4,5-trimethoxybenzyl)-9H-pyrido[3,4-b]indolyl]acetic acid; 2-[4-oxo-5-carboxamido-9-(3,4-methylenedioxybenzyl)-9H-pyrido[3,4-b]indolyl]acetic acid; 2-[4-oxo-5-carboxamido-9-(4-methoxy-3-methylbenzyl)-9H-pyrido[3,4-b]indolyl]acetic acid; 2-[4-oxo-5-carboxamido-9-(3,5-dimethoxybenzyl)-9H-pyrido[3,4-b]indolyl]acetic acid; 2-[4-oxo-5-carboxamido-9-(2,5-dimethoxybenzyl)-9H-pyrido[3,4-b]indolyl]acetic acid; 2-[4-oxo-5-carboxamido-9-(4-ethoxybenzyl)-9H-pyrido[3,4-b]indolyl]acetic acid; 2-[4-oxo-5-carboxamido-9-(cyclohexylmethyl)-9H-pyrido[3,4-b]indolyl]acetic acid; 2-[4-oxo-5-carboxamido-9-(cyclopentylmethyl)-9H-pyrido[3,4-b]indolyl]acetic acid; 2-[4-oxo-5-carboxamido-9-ethyl-9H-pyrido[3,4-b]indolyl]acetic acid; 2-[4-oxo-5-carboxamido-9-(1-propyl)-9H-pyrido[3,4-b]indolyl]acetic acid; 2-[4-oxo-5-carboxamido-9-(2-propyl)-9H-pyrido[3,4-b]indolyl]acetic acid; 2-[4-oxo-5-carboxamido-9-(1-butyl)-9H-pyrido[3,4-b]indolyl]acetic acid; 2-[4-oxo-5-carboxamido-9-(2-butyl)-9H-pyrido[3,4-b]indolyl]acetic acid; 2-[4-oxo-5-carboxamido-9-isobutyl-9H-pyrido[3,4-b]indolyl]acetic acid; 2-[4-oxo-5-carboxamido-9-[2-(1-phenylethyl)]-9H-pyrido[3,4-b]indolyl]acetic acid; 2-[4-oxo-5-carboxamido-9-[3-(1-phenylpropyl)]-9H-pyrido[3,4-b]indolyl]acetic acid; 2-[4-oxo-5-carboxamido-9-[4-(1-phenylbutyl)]-9H-pyrido[3,4-b]indolyl]acetic acid; 2-[4-oxo-5-carboxamido-9-(1-pentyl)-9H-pyrido[3,4-b]indolyl]acetic acid; 2-[4-oxo-5-carboxamido-9-(1-hexyl)-9H-pyrido[3,4-b]indolyl]acetic acid; 4-[(9-benzyl-4-carbamoyl-1,2,3,4-tetrahydrocarbazol-6-yl)oxy]butyric acid; 3-[(9-benzyl-4-carbamoyl-1,2,3,4-tetrahydrocarbazol-6-yl)oxy]propylphosphonic acid; 2-[(9-benzyl-4-carbamoyl-1,2,3,4-tetrahydrocarbazol-6-yl)oxy]methylbenzoic acid; 3-[(9-benzyl-4-carbamoyl-7-n-octyl-1,2,3,4-tetrahydrocarbazol-6-yl)oxy]propylphosphonic acid; 4-[(9-benzyl-4-carbamoyl-7-ethyl-1,2,3,4-tetrahydrocarbazol-6-yl)oxy]butyric acid; 3-[(9-benzyl-4-carbamoyl-7-ethyl-1,2,3,4-tetrahydrocarbazol-6-yl)oxy]propylphosphonic acid; 3-[(9-benzyl-4-carbamoyl-7-ethyl-1,2,3,4-tetrahydrocarbazol-6-yl)oxy]propylphosphonic acid; (S)-(+)-4-[(9-benzyl-4-carbamoyl-7-ethyl-1,2,3,4-tetrahydrocarbazol-6-yl)oxy]butyric acid; 4-[9-benzyl-4-carbamoyl-6-(2-cyanoethyl)-1,2,3,4-tetrahydrocarbazol-6-yl]oxybutyric acid; 4-[9-benzyl-4-carboxamido-7-(2-phenylethyl)-1,2,3,4-tetrahydrocarbazol-6-yl]oxybutyric acid; 4-[9-benzyl-4-carboxamidocarbazol-6-yl]oxybutyric acid; methyl 2-[(9-benzyl-4-carbamoyl-1,2,3,4-tetrahydrocarbazol-6-yl)oxy]methylbenzoate; 4-[9-benzyl-4-carbamoyl-7-(2-cyanoethyl)-1,2,3,4-tetrahydrocarbazol-6-yl]oxybutyric acid; 9-benzyl-7-methoxy-5-cyanomethyloxy-1,2,3,4-tetrahydrocarbazole-4-carboxamide; [9-benzyl-4-carbamoyl-8-methyl-carbazole-5-yl]oxyacetic acid; and [9-benzyl-4-carbamoyl-carbazole-5-yl]oxyacetic acid, or pharmaceutically acceptable salts, solvates, prodrug derivatives, racemates, tautomers, or optical isomers thereof.

Certain embodiments of the methods and compositions provided herein utilize the sPLA₂ inhibitor 3-(2-Amino-1,2-dioxoethyl)-2-ethyl-1-(phenylmethyl)-1H-indol-4-yl)oxy)acetic acid (A-001, also referred to in the art as S-5920 or LY315920) or a salt, solvate, or prodrug thereof. Certain embodiments utilize the sodium salt of A-001. A-001 has the structure:

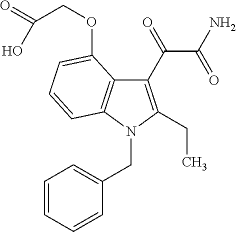

A-001 is a competitive inhibitor of sPLA₂.

Certain embodiments of the methods and compositions provided herein utilize an A-001 prodrug, and in certain of these embodiments the prodrug is a C₁-C₆ alkyl ester, acyloxyalkyl ester, or alkyloxycarbonyloxyalkyl ester of A-001. In certain of these embodiments, the prodrug is A-002 (also referred to in the art as S-3013, LY333013, or varespladib methyl), which has the structure:

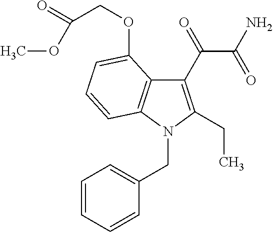

A-002, which has a terminal half-life (t_{1/2}) of approximately ten hours, is rapidly absorbed and hydrolyzed to the active A-001 molecule. One skilled in the art will recognize that other prodrug forms of A-001 may be used in the methods and compositions disclosed herein. One skilled in the art would recognize that any prodrug that is metabolized to the active A-001 molecule would be likely to have similar therapeutic characteristics, and such a skilled artisan could identify such prodrugs with minimal experimentation.

In those embodiments of the compositions and methods disclosed herein that utilize statins, examples of statins that may be used include, but are not limited to, atorvastatin or atorvastatin calcium (marketed as Lipitor® or Torvast®; see, e.g., U.S. Pat. No. 4,681,893 or 5,273,995) and atorvastatin combinations (e.g., atorvastatin plus amlodipine (marketed as Norvasc®), combination marketed as Caduet®, see, e.g., U.S. Pat. No. 6,455,574; atorvastatin plus CP-529414 (marketed as Torcetrapib®); atorvastatin plus APA-01; atorvastatin plus ezetimibe), cerivastatin (marketed as Lipobay® or Baycol®), fluvastatin (marketed as Lescol®; U.S. Pat. No. 4,739,073), lovastatin (marketed as Mevacor® or Altocor®; see, e.g., U.S. Pat. No. 4,231,938), lovastatin combinations (e.g., lovastatin plus Niaspan®, combination marketed as Advicor®), mevastatin, pitavastatin (marketed as Livalo® or Pitava®), pravastatin (marketed as Pravachol®, Mevalotin®, Selektine®, or Lipostat®; see, e.g., U.S. Pat. No. 4,346,227), pravastatin combinations (e.g., pravastatin plus fenofibrate), rosuvastatin (marketed as Crestor®), rosuvastatin combinations (e.g., rosuvastatin plus TriCor®), simvastatin (marketed as Zocor® or Lipex®; see, e.g., U.S. Pat. Nos. 4,444,784; 4,916,239; and 4,820,850), and simvastatin combinations (e.g., simvastatin plus ezetimibe, combination marketed as Vytorin®, see, e.g., U.S. Pat. No. 7,229,982; simvastatin plus Niaspan®, combination marketed as Simcor®; simvastatin plus MK-0524A, combination referred to as MK-0524B), as well as various pharmaceutically acceptable salts, solvates, salts, stereoisomers, prodrugs derivatives, or nitroderivatives of the compounds listed above. In some cases, such as for example with simvastatin, the active form of the statin is a metabolite formed in the body of a subject following administration. In other cases, statins are administered in their active form. In certain embodiments, statins may be administered according to their standard recommended dosage, while in other embodiments statins may be administered lower than the recommended dosage.

In certain embodiments, methods are provided for treating MACEs in a diabetic subject who has previously experienced an ACS event by administering a therapeutically effective amount of one or more sPLA₂ inhibitors alone or in combination with one or more statins. In certain embodiments, the previous ACS event occurred recently, such as for example within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain of these embodiments, the subject has experienced an ACS event within 96 hours of the first administration of the one or more sPLA₂ inhibitors. In certain embodiments, the previously ACS event was diagnosed recently, such as for example within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain of these embodiments, the subject has been diagnosed with an ACS event within 96 hours of the first administration of the one or more sPLA₂ inhibitors. In certain embodiments, administration of one or more sPLA₂ inhibitors and/or statins may result in treatment of one or more specific MACE types (e.g., UA (including UA requiring urgent hospitalization), cardiovascular death, fatal or non-fatal MI, fatal or non-fatal stroke, and/or need for revascularization procedures). In certain of these embodiments, administration of one or more sPLA₂ inhibitors and/or statins results in treatment of UA requiring urgent hospitalization, and in certain of these embodiments treatment of UA refers to a decrease in the occurrence of UA. In other embodiments, MACE treatment is observed across the entire spectrum of MACEs or across a defined subset of MACEs. In certain embodiments, sPLA₂ inhibitors and/or statins are administered in conjunction with one or more pharmaceutically acceptable carriers. In certain embodiments, the one or more sPLA₂ inhibitors include A-001 or a prodrug thereof, and in certain of these embodiments the prodrug thereof is A-002. In certain embodiments, the one or more statins include atorvastatin, rosuvastatin, and/or simvastatin.

In certain embodiments, methods are provided for reducing the likelihood of a MACE in a diabetic subject who has previously experienced an ACS event by administering a therapeutically effective amount of one or more sPLA₂ inhibitors alone or in combination with one or more statins. In certain embodiments, the previous ACS event occurred recently, such as for example within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain of these embodiments, the subject has experienced an ACS event within 96 hours of the first administration of the one or more sPLA₂ inhibitors. In certain embodiments, the previously ACS event was diagnosed recently, such as for example within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain of these embodiments, the subject has been diagnosed with an ACS event within 96 hours of the first administration of the one or more sPLA₂ inhibitors. In certain embodiments, administration of one or more sPLA₂ inhibitors and/or statins may result in a reduced likelihood of one or more specific MACE types (e.g., UA (including UA requiring urgent hospitalization), cardiovascular death, fatal or non-fatal MI, fatal or non-fatal stroke, and/or need for revascularization procedures). In certain of these embodiments, administration of one or more sPLA₂ inhibitors and/or statins results in a reduced likelihood of UA requiring urgent hospitalization. In other embodiments, the reduction in MACE occurrence is observed across the entire spectrum of MACEs or across a defined subset of MACEs. In certain embodiments, sPLA₂ inhibitors and/or statins are administered in conjunction with one or more pharmaceutically acceptable carriers. In certain embodiments, the one or more sPLA₂ inhibitors include A-001 or a prodrug thereof, and in certain of these embodiments the prodrug thereof is A-002. In certain embodiments, the one or more statins include atorvastatin, rosuvastatin, and/or simvastatin.

In certain embodiments, administration of one or more sPLA₂ inhibitors alone or in combination with one or more statins is more effective at treating MACEs than administration of one or more statins alone over a particular time period. Therefore, provided herein in certain embodiments are methods for increasing the effectiveness of one or more statins at treating MACEs in a diabetic post-ACS event subject by administering one or more sPLA₂ inhibitors in combination with statins. In certain embodiments, administration of one or more sPLA₂ inhibitors in combination with one or more statins may be more effective than administration of one or more statins at treating MACEs over a period of 2 weeks, 4 weeks, 6 weeks, 8 weeks, 10 weeks, 12 weeks, 14 weeks, 16 weeks, 20 weeks, 24 weeks, or 28 weeks after the subject experienced an ACS event, was diagnosed as having experienced an ACS event, and/or received the first administration of sPLA₂ inhibitor. This increased effectiveness may result in prevention of MACE occurrence, a decrease in the likelihood of MACE occurrence, a decrease in the severity of MACE occurrence, and/or a delay in MACE occurrence. In certain embodiments, the improvement in MACE treatment may be observed only in one or more specific types of MACE (e.g., UA (including UA requiring urgent hospitalization), cardiovascular death, fatal or non-fatal MI, fatal or non-fatal stroke, and/or need for revascularization procedures). In certain of these embodiments, the improvement is observed in UA requiring urgent hospitalization, and in certain of these embodiments the improvement manifests as a reduction in UA occurrence. In other embodiments, the improvement in MACE treatment is observed across the entire spectrum of MACEs or across a defined set of MACEs. In certain embodiments, administration of one or more sPLA₂ inhibitors alone or in combination with one or more statins may shift the likelihood of MACE occurrence from more severe to less severe forms. For example, administration of sPLA₂ inhibitor alone or in combination with one or more statins may reduce the number of fatal MACEs versus administration of statin alone, but have no effect on the overall number of MACEs.

In certain embodiments, the use of one or more sPLA₂ inhibitors as an adjunct therapy to statin following an ACS event to reduce the risk of MACEs in a diabetic subject is provided. In certain of these embodiments, the one or more sPLA₂ inhibitors include A-001 or a prodrug thereof, and in certain of these embodiments the prodrug thereof is A-002. In certain embodiments, the statin is atorvastatin, rosuvastatin, and/or simvastatin. In certain embodiments, administration of one or more sPLA₂ inhibitors as an adjunct therapy to statins following an ACS event reduces the risk of one or more MACEs including UA including UA requiring urgent hospitalization, cardiovascular death, fatal or non-fatal MI, fatal or non-fatal stroke, and revascularization procedures. In certain embodiments, the first administration of sPLA₂ inhibitor takes place within 24 hours, 24 to 48 hours, 48 to 96 hours, 96 hours to 1 week, 1 to 2 weeks, 2 to 6 weeks, or 6 to 12 weeks of the occurrence or diagnosis of an ACS event. In certain of these embodiments, the first administration of sPLA₂ inhibitor takes place within 96 hours of the occurrence or diagnosis of the ACS event. In certain embodiments, the use of A-002 in combination with any dosage of statin is provided, wherein A-002 is first administered within 96 hours of an ACS event and is administered for up to 16 weeks, and wherein administration results in prevention of UA including UA requiring urgent hospitalization, cardiovascular death, non-fatal MI, or non-fatal stroke. In other embodiments, the use of A-002 in combination with any dosage of atorvastatin or rosuvastatin is provided, wherein A-002 is first administered within 96 hours of an ACS event and is administered for up to 90 days, and wherein administration results in reduction of UA requiring urgent hospitalization.

In certain embodiments, methods are provided for inhibiting inflammation in a diabetic subject who has previously experienced an ACS event by administering a therapeutically effective amount of one or more sPLA₂ inhibitors alone or in combination with one or more statins. In certain of these embodiments, administration of one or more sPLA₂ inhibitors alone or in combination with one or more statins results in a decrease in the level of one or more inflammatory markers, including for example hs-CRP, sPLA₂, and IL-6. In certain embodiments, the previous ACS event occurred recently, such as for example within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain of these embodiments, the subject has experienced an ACS event within 96 hours of the first administration of the one or more sPLA₂ inhibitors. In certain embodiments, the previously ACS event was diagnosed recently, such as for example within 24 hours, 24 to 48 hours, 48 to 96 hours, 96 hours to 1 week, 1 to 2 weeks, 2 to 6 weeks, or 6 to 12 weeks prior to the first administration of the one or more sPLA₂ inhibitors. In certain of these embodiments, the subject has been diagnosed with an ACS event within 96 hours of the first administration of the one or more sPLA₂ inhibitors. In certain embodiments, sPLA₂ inhibitors and/or statins are administered in conjunction with one or more pharmaceutically acceptable carriers. In certain embodiments, the one or more sPLA₂ inhibitors include A-001 or a prodrug thereof, and in certain of these embodiments the prodrug thereof is A-002. In certain embodiments, the one or more statins include atorvastatin, rosuvastatin, and/or simvastatin.

In certain embodiments of the methods provided herein, one or more additional therapeutics used in the treatment of CVD, MACEs, or ACS may be administered to a subject in conjunction with one or more sPLA₂ inhibitors and/or one or more statins. For example, sPLA₂ inhibitors and/or statins may be administered in conjunction with one or more of aspirin, ACE inhibitors, beta-adrenergic blockers, and/or anti-platelet therapy.

In certain embodiments, kits are provided for treating MACEs, including reducing the likelihood of MACEs, in a diabetic subject who has previously experienced an ACS event. These kits comprise one or more sPLA₂ inhibitors, and in certain embodiments the kits further comprise one or more statins. In certain embodiments, the one or more sPLA₂ inhibitors include A-001 or a prodrug thereof, and in certain of these embodiments the prodrug thereof is A-002. In certain embodiments, the one or more statins include atorvastatin, rosuvastatin, and/or simvastatin. In certain embodiments, the kit includes instructions for usage, such as dosage or administration instructions.

In certain embodiments of the methods provided herein, the one or more sPLA₂ inhibitors may be administered via different routes and/or in different forms at different times over the course of treatment. For example, in certain embodiments the one or more sPLA₂ inhibitors may be administered via a parenteral route such as infusion in the hours and days immediately following the ACS event, followed by administration via a different route at later timepoints. These embodiments allow for rapid administration of sPLA₂ inhibitor in the hours and/or days immediately following an ACS event. In addition, they allow for easier administration of the compound to a subject who is incapacitated or partially incapacitated. The form of the drug may vary depending on the administration route being used. For example, in certain embodiments A-001 may be administered via a parenteral route in the early timepoints after an ACS event. At later timepoints, parenteral administration may be phased out and replaced with oral administration of A-002 or another prodrug form of A-001. The phase out from parenteral to oral administration may occur gradually, with parenteral administration being reduced over a series of timepoints while oral administration is simultaneously increased. Alternatively, parenteral administration may be discontinued all at once, and the subject may be switched immediately to a full oral dosage of the drug. In other embodiments, a subject may receive sPLA₂ inhibitors in different forms and/or via different administration routes throughout the entire course of treatment. Administering sPLA₂ inhibitors via a parenteral route in the time period immediately after an ACS event may be advantageous in certain embodiments because it allows for therapeutic blood levels of the drug to be obtained rapidly more. In addition, it allows for blood levels of the drug to be maintained at more steady levels.

In certain embodiments of the methods provided herein wherein one or more sPLA₂ inhibitors and one or more statins are administered to a subject, the one or more sPLA₂ inhibitors and one or more statins may be administered separately, i.e., in separate compositions. In these embodiments, the one or more sPLA₂ inhibitors and one or more statins may be administered simultaneously or sequentially. Further, one or more sPLA₂ inhibitors and one or more statins may be administered at different times, and one compound may be administered more frequently than another. In certain embodiments wherein one or more sPLA₂ inhibitors and one or more statins are given in multiple administrations, one or both may be administered anywhere from one or more times per day to once every week, once every month, or once every several months. In certain of these embodiments, the one or more sPLA₂ inhibitors and/or one or more statins may be administered once a day, twice a day, or three times a day. Alternatively, the one or more sPLA₂ inhibitors and one or more statins may be administered continuously or semi-continuously, such as for example by intravenous infusion. In certain embodiments, administration of one or more sPLA₂ inhibitors and one or more statins may begin at the same time. In these embodiments, administration of sPLA₂ inhibitor and statin may begin within a certain time period after an ACS event or diagnosis of an ACS event, such as for example within 96 hours. In other embodiments, administration of one or more sPLA₂ inhibitors and one or more statins may begin at different times. In these embodiments, either compound may be administered first. For example, one or more sPLA₂ inhibitors may be administered first within a certain time period after an ACS event or diagnosis of an ACS event, such as for example within 96 hours of the event, with administration of statin beginning at a later timepoint. Alternatively, administration of one or more statins may begin before administration of one or more sPLA₂ inhibitors. In these embodiments, the subject may already have been on statin prior to the ACS event. When the subject was already on statin prior to the ACS event, statin administration after the event may continue at the same dosage and administration interval as before the ACS event. Alternatively, the dosage and/or administration interval of the statin may be adjusted after the ACS event. In addition, the specific statin being administered may be changed following the ACS event. For example, a subject that was receiving rosuvastatin prior to an ACS event may switch to atorvastatin following the event, or vice versa.

In other embodiments, one or more sPLA₂ inhibitors and one or more statins may be administered as part of a single composition. Provided herein in certain embodiments are such compositions, as well as kits comprising these compositions and the use of one or more sPLA₂ inhibitors and one or more statins in producing these compositions. In those embodiments wherein one or more sPLA₂ inhibitors and one or more statins are administered to a subject as a single composition, the composition may be administered on a one-time basis or in multiple administrations. In those embodiments wherein the composition is given in multiple administrations, it may be administered anywhere from one or more times per day to once every week, once every month, or once every several months. In certain of these embodiments, the composition may be administered once a day, twice a day, or three times a day. Alternatively, the composition may be administered continuously or semi-continuously, such as for example by parenteral administration. In certain embodiments, the composition may comprise one or more additional CVD therapeutics, such as for example aspirin, ACE inhibitors, beta-adrenergic blockers, and/or anti-platelet therapy.

One or more sPLA₂ inhibitors, one or more statins, or compositions comprising one or more sPLA₂ inhibitors and one or more statins may be administered on a one-time basis, continuously, or at set intervals over a particular time period. In those embodiments wherein the compounds are administered over a particular time period, the time period may be determined in advance and may be measured in weeks or days. For example, in certain embodiments one or more sPLA₂ inhibitors may be administered at set intervals over a 2 week, 4 week, 6 week, 8 week, 10 week, 12 week, 14 week, 15 week, 16 week, 17 week, 18 week, 19 week, 20 week, 24 week, or 28 week period. In certain of these embodiments, one or more sPLA₂ inhibitors are administered for up to 16 weeks. In certain embodiments, one or more sPLA₂ inhibitors may be administered for up to 70 days, up to 80 days, up to 90 days, up to 100 days, up to 110 days, up to 112 days, up to 115 days, or up to 120 days. In certain of these embodiments, one or more sPLA₂ inhibitors are administered for up to 112 days.

In certain embodiments, methods are provided for preventing UA requiring urgent hospitalization in a diabetic subject who has experienced an ACS event within the past 96 hours by administering A-001 or a salt, solvate, or prodrug thereof and any statin at regular intervals for a maximum of 16 weeks. In certain of these embodiments the prodrug thereof is A-002. In certain embodiments, the interval at which A-001 or a salt, solvate, or prodrug thereof is administered is once, twice, or three times daily. In certain embodiments, A-001 or a salt, solvate, or prodrug thereof is administered continuously or semi-continuously.

Compositions comprising one or more sPLA₂ inhibitors and/or one or more statins may be administered by any administration pathway known in the art, including but not limited to oral, aerosol, enteral, nasal, ophthalmic, parenteral, or transdermal (e.g., topical cream or ointment, patch). “Parenteral” refers to a route of administration that is generally associated with injection, such as for example bolus injection or continuous or semi-continuous infusion. Parenteral administration may be accomplished by a variety of pathways, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. One or more sPLA₂ inhibitors, one or more statins, or combined sPLA₂ inhibitor/statin compositions as described herein may be administered in any pharmaceutically acceptable form, including for example in the form of a solid, liquid solution, suspension, emulsion, dispersion, micelle, or liposome. Preparations for injection may include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile emulsions. The solutions may be either aqueous or nonaqueous. In certain embodiments, the compositions may comprise one or more pharmaceutically acceptable carriers or may be administered in conjunction with one or more pharmaceutically acceptable carriers.

In certain embodiments, pharmaceutical compositions comprising one or more sPLA₂ inhibitors or one or more sPLA₂ inhibitors and one or more statins may be formed into oral dosage units, such as for example tablets, pills, or capsules. Such an oral dosage unit may comprise the active ingredients (e.g., A-002 and atorvastatin) and one or more pharmaceutically acceptable carriers. In certain embodiments, pharmaceutical compositions comprising one or more sPLA₂ inhibitors and/or one or more statins may be administered via a time release delivery vehicle, such as for example a time release oral dosage unit. A “time release vehicle” as used herein refers to any delivery vehicle that releases active agent (e.g., A-002 and atorvastatin) at some time after administration or over a period of time following administration rather than immediately upon administration. Time release may be obtained by a coating on the vehicle that dissolves over a set timeframe following administration. In certain embodiments, the time release vehicle may comprise multiple layers of coating alternated with multiple layers of active ingredients, such that each layer of coating releases a certain volume of active ingredients as it dissolves. In other embodiments, one or more sPLA₂ inhibitors and/or one or more statins may be administered via an immediate release delivery vehicle.

A therapeutically effective amount of one or more sPLA₂ inhibitors and/or one or more statins may be determined for each compound individually. For example, statins may be administered or included in a pharmaceutical composition at a dosage that is well known in the art. In these embodiments, statins may be administered according to the manufacturer instructions for the particular statin. In certain of these embodiments, a particular statin may be administered at a dosage ranging from about 5 mg to about 80 mg. For example, in those embodiments wherein the statin is atorvastatin, simvastatin, or rosuvastatin, the statin may be administered at a dosage of about 5, 10, 20, 40, 60, or 80 mg. One skilled in the art will recognize that in those embodiments wherein one or more statins are combined with one or more sPLA₂ inhibitors in a single composition, the amount of statin that constitutes a therapeutically effective amount may be different than the amount of statin that constitutes a therapeutically effective amount when administered alone due to, for example, interactions between the statin and the sPLA₂ inhibitor. For example, the effective dosage of a statin for use in combination therapy may be lower than the effective dosage for the statin when administered alone. Likewise, the therapeutically effective amount of an sPLA₂ inhibitor may be lower when administered in conjunction with a statin than when the sPLA₂ inhibitor is administered alone. In these situations, one skilled in the art will readily be able to determine a therapeutically effective amount for the combination using methods well known in the art. In certain embodiments, a therapeutically effective amount of one or more sPLA₂ inhibitors for use either alone or in combination with one or more statins is about 25 to about 5,000 mg/dose, and in certain of these embodiments a therapeutically effective amount may be from about 50 to about 1,000 mg/dose. The therapeutically effective amount of an sPLA₂ inhibitor or statin may change over the course of administration. For example, dosages may be increased or decreased as necessary in the weeks following an ACS event based on therapeutic response, side effects, and/or other factors.

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention. It will be understood that many variations can be made in the procedures herein described while still remaining within the bounds of the present invention. It is the intention of the inventors that such variations are included within the scope of the invention.

### EXAMPLES

### Example 1

### Effect of A-002 Plus Statin on Major Adverse Cardiac Events in Subjects with Diabetes

625 adult (18 years of age or older) human subjects who had recently experienced an index ACS event (UA, NSTEMI, or STEMI) were randomized to receive placebo or A-002 at 500 mg once daily via oral administration in a double-blinded manner. A-002 was delivered in the form of two 250 mg tablets. In addition, all subjects received atorvastatin at 80 mg once daily via oral administration of a single tablet. Subtypes of index ACS events were similarly distributed between the A-002/atorvastatin group and the atorvastatin only group.

Subjects were randomized within 96 hours of hospital admission for an index ACS event, or within 96 hours of index ACS event diagnosis if already hospitalized. Prior to randomization, subjects were screened for pertinent medical history, and baseline levels of LDL and hs-CRP were measured. Baseline sPLA₂ levels were also measured in a random subset of subjects. Percutaneous revascularization, if required or planned for a particular subject, was performed prior to randomization.

In addition an index ACS event, all subjects exhibited one or more of the following: diabetes, a BMI of 25 kg/m² or greater; serum hs-CRP levels of 2 mg/L or greater if diagnosed with NSTEMI or STEMI or 3 mg/L or greater if diagnosed with UA; or at least three characteristics of metabolic syndrome (waist circumference greater than 102 cm (male) or 88 cm (female), serum TG levels of 150 mg/dL (1.7 mmol/L) or greater, HDL levels less than 40 mg/dL (1.0 mmol/L) (male) or 50 mg/dL (1.3 mmol/L) (female), blood pressure of 130/85 mm Hg or greater, or plasma glucose of 110 mg/dL (6.1 mmol/L) or greater. Subjects were excluded if they were receiving statin therapy at maximum recommended or tolerated dosage (i.e., 40-80 mg QD for atorvastatin, fluvastatin, lovastatin, pravastatin, or simvastatin, or 20-40 mg QD rosuvastatin) at the time of the index ACS event. During the trial, subjects were prevented from using any lipid-lowering therapy other than 80 mg atorvastatin and/or A-002.

For purposes of determining whether a subject had experienced an index ACS event, subjects were defined as having UA if they exhibited: 1) chest pain or angina occurring at rest or with minimal exertion, lasting longer than ten minutes, and consistent with myocardial ischemia within 24 hours prior to hospitalization; 2) an ECG reading with new or dynamic ST or T wave changes of 1 mm or greater, horizontal or down sloping ST segment depression not previously present in at least two contiguous leads, or new wall motion or reversible perfusion abnormalities; and 3) cardiac troponin I levels of 0.1 ng/ml or greater but less than upper limit of normal (ULN) or cardiac troponin T levels of 0.2 ng/ml or greater. Subjects were defined as having NSTEMI if they exhibited: 1) no ECG changes, ST depression, or T wave changes (i.e., no new Q waves on serial ECGs) and 2) an increase in cardiac troponin greater than the local limit for the definition of MI or an increase in CK-MB isoenzyme greater than ULN. Subjects were defined as having STEMI if they exhibited: 1) persistent ST or T wave changes or ST segment elevation of at least 2 mm in two contiguous leads and persisting longer than 15 minutes, and 2) an increase in cardiac troponin greater than the local limit for the definition of MI or an increase in CK-MB greater than ULN.

Individual subjects received treatment until all subjects had been treated for a minimum of 24 weeks or until the occurrence of a MACE. For purposes of this study, MACEs included all-cause mortality, non-fatal MI, documented UA requiring urgent hospitalization, revascularization occurring 60 days or more after the initial index ACS event, and non-fatal stroke. Subjects were evaluated at various timepoints after randomization and at study completion. Each evaluation included measurement of serum LDL levels and recordation of any MACEs or less severe adverse events occurring since the previous evaluation. In addition, certain evaluation periods included measurement of one or more of hs-CRP, sPLA₂, IL-6, and/or other biomarker levels, vital signs, weight, and/or waist circumference. All active subjects (i.e., those who did not have a MACE or withdraw early) received a final evaluation when treatment ended. This final evaluation included at least a complete physical examination, a 12-lead ECG reading, measurement of LDL, hs-CRP, sPLA₂, and IL-6 levels, and recordation of any MACEs during the study period.

Subjects from the ITT population receiving A-002 plus atorvastatin exhibited a greater decrease in mean serum LDL levels than subjects receiving atorvastatin only. This difference was observed at the earliest timepoint measured (2 weeks) and at all subsequent timepoints (4, 8, and 16 weeks). Administration of A-002 plus atorvastatin also resulted in a greater percentage of subjects reaching LDL target levels of 100 mg/dl or less, 70 mg/dl or less, and 50 mg/dl or less. Overall, these results established that A-002 in combination with statin reduces LDL levels more rapidly and to a greater overall extent than statin alone in an unstable post-ACS event population.

Subjects from the ITT population receiving A-002 plus atorvastatin also exhibited a greater decrease in mean and median levels of the inflammatory markers hs-CRP, sPLA₂, and IL-6 at weeks 2, 4, 8, and 16 than subjects receiving atorvastatin only. In addition, A-002 plus atorvastatin was more effective at helping patients reach composite target levels of less than 70 mg/dl LDL and less than 3 mg/L hs-CRP than atorvastatin alone.

In order to determine whether A-002 was capable of decreasing inflammation in a post-ACS population with exceptionally high inflammation levels, inflammatory marker levels were evaluated in a subpopulation with diabetes. As with the ITT population, A-002 plus atorvastatin decreased hs-CRP and IL-6 levels in this subpopulation to a greater extent than atorvastatin alone.

TABLE 1
Effect of A-002 administration on serum hs-CRP levels in diabetic
subpopulation

| | | | | Difference |
|---|---|---|---|---|
| | | | | between A- |
| | | | Placebo | 002/atorvastatin |
| | | A-002 plus | (atorvastatin | and placebo |
| | | atorvastatin | only) | (p-value) |
| Week 2 | # of subjects | 46 | 55 | |
| | % change in | −58.8% | −11.0 | 47.8% |
| | median [hs- | | | (0.0004) |
| | CRP] from | | | |
| | baseline | | | |
| Week 4 | # of subjects | 69 | 70 | |
| | % change in | −83.0% | −51.1% | 31.9% |
| | median [hs- | | | (0.0013) |
| | CRP] from | | | |
| | baseline | | | |
| Week 8 | # of subjects | 70 | 63 | |
| | % change in | −82.8% | −67.6% | 15.2% |
| | median [hs- | | | (0.0299) |
| | CRP] from | | | |
| | baseline | | | |
| Week 16 | # of subjects | 67 | 65 | |
| | % change in | −83.6% | −72.4% | 11.2% |
| | median [hs- | | | (0.0776) |
| | CRP] from | | | |
| | baseline | | | |
| Week 24 | # of subjects | 50 | 53 | |
| | % change in | −89.5% | −76.1% | 13.4% |
| | median [hs- | | | (0.0311) |
| | CRP] from | | | |
| | baseline | | | |

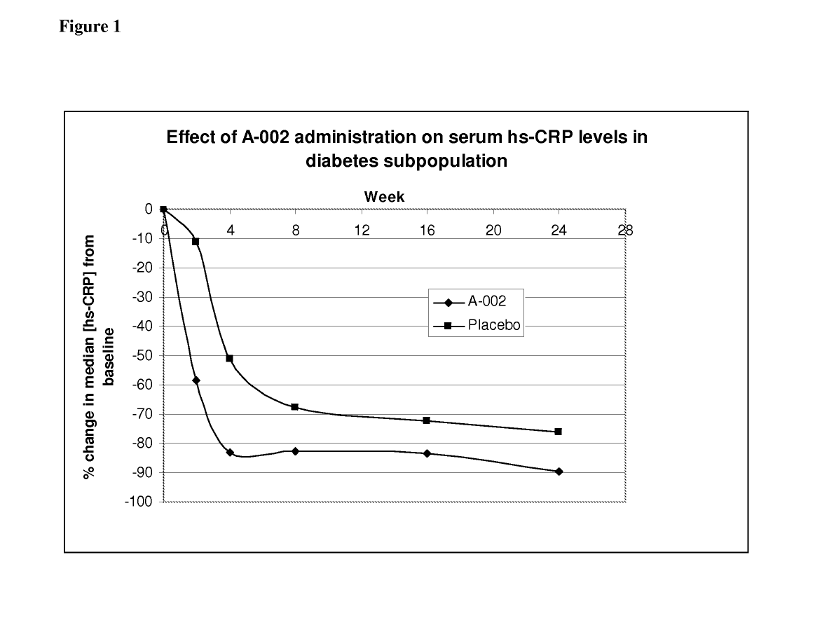

All groups in a diabetic subpopulation exhibited a decrease in median hs-CRP levels from baseline at all timepoints measured. Subjects receiving A-002 plus atorvastatin exhibited a greater percent decrease in median hs-CRP levels than subjects receiving atorvastatin alone at all timepoints. These results, which are further summarized in FIG. 1, indicate that A-002 plus statin is capable of reducing inflammation following an ACS event in subjects with diabetes.

TABLE 2
Effect of A-002 administration on serum IL-6 levels in diabetes
subpopulation

| | | | | Difference |
|---|---|---|---|---|
| | | | | between A- |
| | | | Placebo | 002/atorvastatin |
| | | A-002 plus | (atorvastatin | and placebo |
| | | atorvastatin | only) | (p-value) |
| Week 2 | # of subjects | 48 | 54 | |
| | % change in | −21.8% | +3.5% | 25.4% |
| | median [IL-6] | | | (0.0019) |
| | from baseline | | | |
| Week 4 | # of subjects | 55 | 59 | |
| | % change in | −47.1% | −23.9% | 13.2% |
| | median [IL-6] | | | (0.0250) |
| | from baseline | | | |
| Week 8 | # of subjects | 51 | 51 | |
| | % change in | −48.8% | −40.4% | 8.8% |
| | median [IL-6] | | | (0.0903) |
| | from baseline | | | |

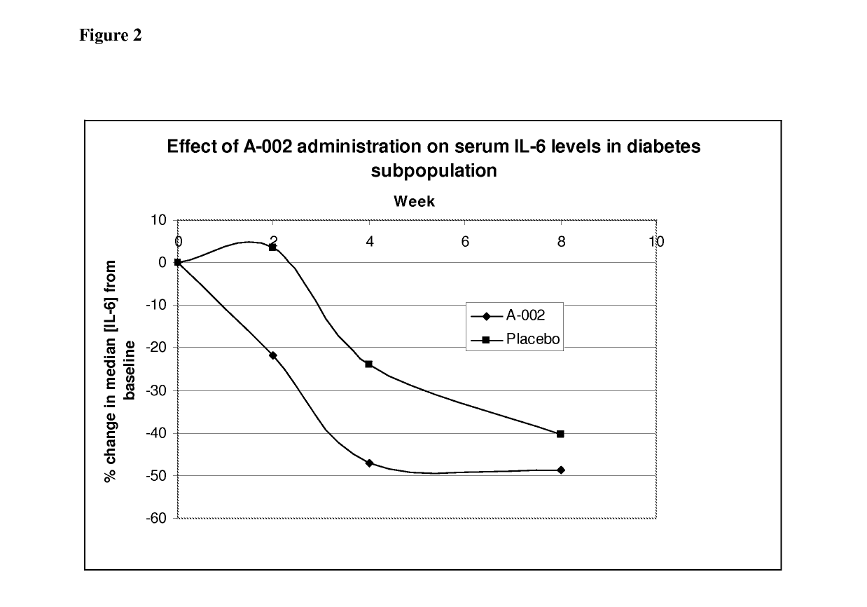

Diabetic subjects receiving atorvastatin alone exhibited an increase in median IL-6 levels from baseline at week 2, followed by a decrease at weeks 4 and 8. Subjects receiving A-002 plus atorvastatin exhibited a decrease in median IL-6 levels from baseline at all timepoints. The greatest difference between subjects receiving A-002 plus atorvastatin versus subjects receiving atorvastatin alone was seen at weeks 2 and 4. These results, which are further summarized in FIG. 2, confirm that A-002 plus statin is capable of reducing inflammation after an ACS event in subjects with diabetes.

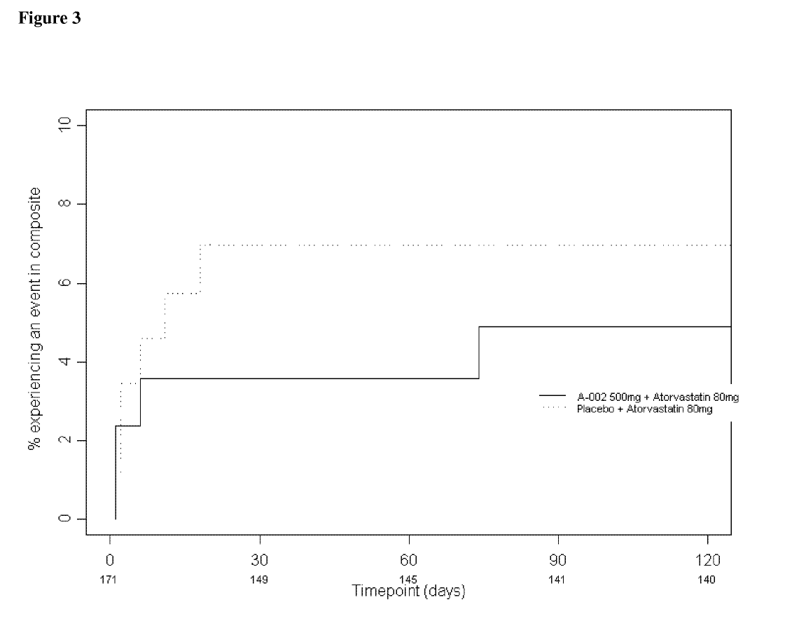

In addition to lowering LDL and inflammatory marker levels in the ITT population, A-002 plus atorvastatin was found to decrease MACE occurrence versus atorvastatin alone. At 16 weeks, only 4.2% of subjects receiving A-002 plus atorvastatin experienced a MACE, versus 6.1% for subjects receiving atorvastatin only. In order to determine whether A-002 plus atorvastatin could also reduce MACE occurrence in a diabetic subpopulation, 171 subjects identified as having diabetes during randomization were evaluated for MACE occurrence at week 16. The high baseline levels of inflammation associated with diabetes makes this subpopulation particularly vulnerable to MACEs in the period following an ACS event. MACE occurrence at 0 to 120 days is shown in FIG. 3, and MACE occurrence at 16 weeks is summarized in Table 3.

TABLE 3
Cumulative effect of A-002 administration on MACEs in diabetic
subpopulation at 16 weeks

| | A-002 plus | Placebo |
|---|---|---|
| | atorvastatin | (atorvastatin only) |
| Population (n) | 84 | 87 |
| Total MACEs | 4 (4.8%) | 6 (6.9%) |
| UA requiring | 0 (0.0%) | 3 (3.4%) |
| hospitalization | | |
| MI | 1 (1.2%) | 0 (0.0%) |
| Stroke | 0 (0.0%) | 0 (0.0%) |
| Death | 3 (3.6%) | 3 (3.4%) |
| Revascularization ≧ | 0 (0.0%) | 0 (0.0%) |
| 60 days | | |

Diabetic subjects receiving A-002 plus atorvastatin experienced fewer total MACEs than subjects receiving atorvastatin only at 16 weeks (4.8% versus 6.9%). As set forth in Table 3, there was no difference between the A-002 and placebo groups with regard to stroke, death, and revascularization, and subjects receiving A-002 actually experienced one more non-fatal MI than placebo subjects. Therefore, the difference in overall MACE occurrence was driven by the difference in UA. None of the subjects receiving A-002 had experienced UA requiring hospitalization at 16 weeks, whereas 3.4% of the placebo group had experienced UA. Therefore, these results show that administration of A-002 plus atorvastatin significantly reduces the likelihood of experiencing UA during the 16 weeks following an index ACS event in subjects with diabetes.

As stated above, the foregoing is merely intended to illustrate various embodiments of the present invention. The specific modifications discussed above are not to be construed as limitations on the scope of the invention. It will be apparent to one skilled in the art that various equivalents, changes, and modifications may be made without departing from the scope of the invention, and it is understood that such equivalent embodiments are to be included herein. All references cited herein are incorporated by reference as if fully set forth herein.

### REFERENCES

1. Boekholdt et al. 2005. Arterioscler Thromb Vasc Biol 25:839-846.
2. Bostrom et al. 2007. Arterioscler Thromb Vasc Biol 27:600-606.
3. Camejo et al. 1998. Atherosclerosis 139:205-222.
4. Cannon et al. 2004. N Engl J Med 350:1495.
5. Chait, A., et al. 2005. J Lipid Res 46:389-403.
6. Elinder et al. 1997. Arterioscler Thromb Vasc Biol 17:2257-2263.
7. Hakala et al. 2001. Arterioscler Thromb Vasc Biol 21:1053-1058.
8. Hartford et al. 2006. J Cardiol 108:55-62.
9. Hurt-Camejo et al. 1997. Arterioscler Thromb Vasc Biol 17:300-309.
10. Ivandic et al. 1999. Arterioscler Thromb Vasc Biol 19:1284-1290.
11. Kimura-Matsumoto et al. 2008. Atherosclerosis 196:81-91.
12. Kugiyama et al. 1999. Circulation 100:1280-1284.
13. Liu et al. 2003. Eur Heart J 24:1824-1832.
14. Mallat et al. 2005. J Am Coll Cardiol 46:1249-1257.
15. Mallat et al. 2007. Arterioscler Thromb Vasc Biol 27:1177-1183.
16. Menschikowski et al. 1995. Atherosclerosis 118:173-181.
17. Nijmeijer et al. 2002. Cardiovasc Res 53:138-146.
18. Pruzanski et al. 1998. J Lipid Res 39:2150-2160.
19. Ridker et al. 2005. N Engl J Med 352:20-28.
20. Ridker et al. 2008. N Engl J Med 359:2195.
21. Rosengren, B., et al. 2006. Arterioscler Thromb Vasc Biol 26:1579-1585.
22. Sartipy et al. 1999. J Biol Chem 274:25913-25920.
23. Schwartz et al. 2001. JAMA 285:1711-1718.
24. Schwartz & Olsson. 2005. Am J Cardiol 96:45 F-53F.
25. Serruys et al. 2008. Circulation 118:1172-1182.
26. Tietge et al. 2000. J Biol Chem 275:10077-10084.

1. A method of decreasing the likelihood of a major adverse cardiac event (MACE) in a diabetic subject that has previously experienced an ACS event comprising administering to said subject a therapeutically effective amount of 3-(2-Amino-1,2-dioxoethyl)-2-ethyl-1-(phenylmethyl)-1H-indol-4-yl)oxy)acetic acid or a pharmaceutically acceptable salt, solvate, or prodrug thereof and a therapeutically effective amount of one or more statins.

2. (canceled)

3. A method of inhibiting inflammation in a diabetic subject who has previously experienced an acute coronary syndrome (ACS) event comprising administering a therapeutically effective amount of 3-(2-Amino-1,2-dioxoethyl)-2-ethyl-1-(phenylmethyl)-1H-indol-4-yl)oxy)acetic acid or a pharmaceutically acceptable salt, solvate, or prodrug thereof and a therapeutically effective amount of one or more statins.

4. The method of claim 1 or 3, wherein said prodrug is selected from the group consisting of a C₁-C₆ alkyl ester prodrug, an acyloxyalkyl ester prodrug, and an alkyloxycarbonyloxyalkyl ester prodrug.

5. The method of claim 4, wherein said C₁-C₆ alkyl ester is [[3-(2-Amino-1,2-dioxoethyl)-2-ethyl-1-(phenylmethyl)-1H-indol-4-yl]oxy]acetic acid methyl ester.

6. The method of claim 1 or 3, wherein said one or more statins are selected from the group consisting of atorvastatin, rosuvastatin, simvastatin, lovastatin, pravastatin, cerivastatin, fluvastatin, mevastatin, and pitavastatin, and a statin combination drug.

7. The method of claim 1, wherein said MACE is selected from one or more of the group consisting of unstable angina requiring urgent hospitalization, cardiovascular death, non-fatal myocardial infarction, and non-fatal stroke.

8. (canceled)

9. The method of claim 1 or 3, wherein the first administration of 3-(2-Amino-1,2-dioxoethyl)-2-ethyl-1-(phenylmethyl)-1H-indol-4-yl)oxy)acetic acid or a pharmaceutically acceptable salt, solvate, or prodrug thereof takes place within 96 hours of the occurrence or diagnosis of said ACS event.

10. The method of claim 1 or 3, wherein said administration of 3-(2-Amino-1,2-dioxoethyl)-2-ethyl-1-(phenylmethyl)-1H-indol-4-yl)oxy)acetic acid or a pharmaceutically acceptable salt, solvate, or prodrug thereof occurs one or more times daily for a maximum of 16 weeks.

11. The method of claim 3, wherein said administration of 3-(2-Amino-1,2-dioxoethyl)-2-ethyl-1-(phenylmethyl)-1H-indol-4-yl)oxy)acetic acid or a pharmaceutically acceptable salt, solvate, or prodrug thereof and one or more statins results in a decrease of one or more inflammatory markers selected from the group consisting of sPLA₂, hs-CRP, and IL-6.

12. The method of claim 1 or 3, wherein 3-(2 Amino-1,2-dioxoethyl)-2-ethyl-1-(phenylmethyl)-1H-indol-4-yl)oxy)acetic acid or a pharmaceutically acceptable salt, solvate, or prodrug thereof.

13. A kit for use in reducing the likelihood of a major adverse cardiac event (MACE) in a diabetic subject who has previously experienced an acute coronary syndrome (ACS) event comprising administering a therapeutically effective amount of 3-(2-Amino-1,2-dioxoethyl)-2-ethyl-1-(phenylmethyl)-1H-indol-4-yl)oxy)acetic acid or a pharmaceutically acceptable salt, solvate, or prodrug thereof and a therapeutically effective amount of one or more statins.

14. The kit of claim 13, wherein said prodrug is selected from the group consisting of a C₁-C₆ alkyl ester prodrug, an acyloxyalkyl ester prodrug, and an alkyloxycarbonyloxyalkyl ester prodrug.

15. The kit of claim 14, wherein said C₁-C₆ alkyl ester is [[3-(2-Amino-1,2-dioxoethyl)-2-ethyl-1-(phenylmethyl)-1H-indol-4-yl]oxy]acetic acid methyl ester.

16. The kit of claim 13, wherein said one or more statins are selected from the group consisting of atorvastatin, rosuvastatin, simvastatin, lovastatin, pravastatin, cerivastatin, fluvastatin, mevastatin, and pitavastatin, and a statin combination drug.
